(19) United States
(12) Patent Application Publication
(10) Pub. No.: US 2025/0241693 A1
(43) Pub. Date: Jul. 31, 2025

(54) SURGICAL BONE PLATE SYSTEM WITH FASTENER INDUCED ADAPTIVE FIT

(71) Applicant: J.M. Longyear Manufacturing, LLC dba Able Medical Devices, Marquette, MI (US)

(72) Inventors: Tyler J. Losinski, Negaunee, MI (US); Katie S. Barron, Negaunee, MI (US); Wade C. DePas, Negaunee, MI (US); Peter J. Didyk, Northville, MI (US)

(21) Appl. No.: 19/038,240

(22) Filed: Jan. 27, 2025

Related U.S. Application Data
(60) Provisional application No. 63/625,376, filed on Jan. 26, 2024.

(51) Int. Cl. A61B 17/80; A61B 17/17; A61B 90/00

(52) U.S. Cl. CPC A61B 17/8061; A61B 17/1728; A61B 17/1792; A61B 17/808; A61B 17/8085; A61B 2090/061; A61B 2090/067

(57) ABSTRACT

A bone plate system to fixate bone portions comprises a polymeric bone plate incorporating a 1× n aperture array of fastener apertures and a plurality of fasteners. The fastener apertures are undersized relative to the fasteners. The fasteners are made from a harder material than the at least one polymeric bone plate material in which the apertures are formed. The polymeric bone plate has a native configuration and an induced multi-curved configuration. The induced multi-curved configuration includes increased curvature comprising at least two of increased bending curvature, increased twisting curvature, and/or increased lateral curvature relative to the native configuration. Inserting the fasteners into the fastener apertures volumetrically displaces portions of the at least one bone plate material in a manner effective to induce the polymeric bone plate to transition from the native configuration to the multi-curved configuration.

### PRIORITY

This application claims the benefit of U.S. Provisional Patent Application No. 63/625,376, filed Jan. 26, 2024, the disclosure of which is incorporated in its entirety herein by reference.

### FIELD OF THE DISCLOSURE

This disclosure relates to surgical bone plate systems including surgical bone plates and fasteners useful to fixate injured bones and, more particularly, to surgical bone plates and fasteners used for bone fixation, wherein insertion of the heads of the fasteners into undersized fastener apertures of the bone plates induces increased curvature in the bone plates with respect to at least two of bending curvature, twisting curvature, and lateral curvature.

### BACKGROUND OF THE DISCLOSURE

Surgical bone plate systems including surgical bone plates and surgical fasteners, such as screws, are used for the fixation of injured bone tissues. Fixation helps an injured bone heal in proper alignment and orientation to allow bone healing. A bone plate often is made from metal or polymeric material. Fasteners often are made from metal materials.

Bone plate systems are useful to help fractured bones heal properly. In this context, the bone plate spans a bone fracture and is attached to the bone portions on each side of a fracture in order to hold the bone portions in proper position and to allow healing to occur. In many instances, bone plate fasteners in the form of screws are used to hold the bone plate to the underlying bone portions. Bone plates have fastener apertures through which the fasteners are inserted and fastened to the bone. The apertures in the bone plate are positioned on both sides of the fracture so that the bone plate can be secured to the bone segments on each side of the fracture.

Bone plates are used with respect to a wide range of injured bones, including the jaw, skull, spine, arm bones, leg bones, sternum, and rib bones. Bones tend to be curved to accommodate complex organic geometry, and so the bone plates have to be curved to match the underlying boney structure being stabilized. In the bone plate field, curvature can be expressed as the inverse of the radius of curvature, often in units of m⁻¹. Most human bones have a radius of curvature in the range from 0 m⁻¹ to 20 m⁻¹. The value of 0 m⁻¹ indicates a flat bone with no curvature. Curvature increases with increasing curvature value. For example, a bone with a curvature of 10 m⁻¹ has a higher degree of curvature than a bone with a curvature of 4 m⁻¹.

In the field of bone plates, there are three main types of bone curvature according to industry terminology. A first type is curvature having an out of plane radius, also referred to herein as bending. A second type of curvature is longitudinal twist, also referred to herein as twisting. A third type of curvature is curvature having an in-plane radius, also referred to herein as lateral curvature. Bone surfaces, such as rib bones, may have two or more of these kinds of curvature, or even all three types of curvature.

The three kinds of curvature can be described with respect to a Cartesian coordinate system having mutually perpendicular x, y, and z axes. For example, consider a reference system in which the x-axis is a horizontal axis, the y-axis is a horizontal axis that is perpendicular to the x-axis, the z-axis is a vertical axis that is perpendicular to the y axis and the x axis, and the x, y, and z axes intersect at an origin. If a bone plate is placed in the xy plane of this system so that the bone plate length extends along the x-axis, then bending or out of plane radius curvature is curvature of the bone plate about an axis parallel to the y axis, longitudinal twist or twisting curvature is rotation of the bone plate about an axis parallel to the x axis, and in-plane radius or lateral curvature is curvature around an axis parallel to the z axis. Bone plates may need all three kinds of curvature in order to match the curvature of the underlying bone portions being fixated.

Bone plates can be supplied in a substantially flat configuration and then can be formed into a matching degree of curvature with the underlying bone during a surgical fixation procedure. Bone plates can be flexed into position and screwed to the bone. Interoperative flexing without needing bending tools save procedural time because the bone plate can be conformed to the bone more quickly than if the bone plate has to be conformed in stepwise increments as might be associated with some types of bone plates. However, a flexed bone plate may be under spring-back forces that can exert undue stresses on the bone plate material, the engagement of the bone plate material with the fasteners, and/or the engagement of the fasteners to the bone.

As an alternatively to merely flexing a bone plate into conformity, bone plates can be progressively and permanently contoured during surgery, but this is time consuming and difficult to get an exact match to the bone. Rib bones, in particular, are highly curved and vary widely in degree of curvature from patient to patient, from rib to rib, and along the length of a single rib.

In order to reduce spring-back forces, bone plates that are flexed into position can be pre-curved to make it easier to flex the bone plate to match the underlying boney structure and reduce spring-back forces and procedural time. This can reduce the amount of stress in the installed bone plate. For example, a bone plate with a pre-formed curvature of 5 m⁻¹ would only have to be flexed an additional 5 m⁻¹ to match a bone with a degree of curvature of 10 m⁻¹. In contrast, a flexible bone plate with no initial curvature would have to be flexed the entire 10 m⁻¹ to match the bone. The pre-curved bone plate would have an easier adaptive fit and less flex stresses after fixation.

Rib bones, in particular, are highly curved and vary widely in degree of curvature from patient to patient, from rib to rib, and along the length of a single rib. Also, rib bones not only bend, but also can twist along their length.

A commercially available thoracic bone fixation system designed for sternum fixation and sold under the tradename VALKYRIE® (Able Medical Devices, Gwinn, MI, USA) includes bone plates having fastener apertures that are undersized relative to the accompanying fasteners. The fasteners and fastener apertures of this system are characterized by a volumetric displacement to plate thickness ratio (defined below), a volumetric displacement to plate aperture diameter ratio (defined below), and a volumetric displacement to screw head diameter ratio (defined below) of 0.49 mm², 0.38 mm², and 0.26 mm², respectively. Installation of the fasteners in linear bone plates of the system induce bending curvature into some bone plates of this system, but not twisting curvature. The system includes a bone plate having fastener apertures arranged in a 1×4 fastener aperture array. Inserting the oversized fasteners into the apertures of the 1×4 bone plate does not induce twisting curvature. The system further includes a 1×16 array of fastener apertures in a zig zag array with two fastener apertures per segment of the zig zag geometry. Inserting fasteners into this zig zag bone plate induces de minimis if any twisting curvature due to the low number of fastener apertures in each zig zag segment.

U.S. Pat. Pub. 2021/0267651 describes a bone plate system including bone plates and screw fasteners. The screw fasteners have tapered and threaded heads and threaded shafts. FIGS. 13 to 15 and FIGS. 57-59 show linear bone plates with a 1×4 array of fastener apertures deployed on curves. FIGS. 22-24 show a bone plate with a 1×16 array of fastener apertures in a zig zag array with two fastener apertures per segment of the zig zag geometry, but none of the array segments has 6 or more fastener apertures. FIGS. 63-65 show a Y-shaped bone plate in which a portion of the bone plate include two 1×4 linear arrays of fastener apertures deployed on a curve, wherein the two arrays are joined to form the Y-shape. The Valkyrie system described above also includes such bone plates. Inserting fasteners into the bone plates of the Valkyrie system having these similar geometries induces de minimis if any twisting curvature.

Pre-curved bone plates have drawbacks. Unless stored flat, packaging needs to be bigger to accommodate the pre-curve. Hence, they store inefficiently. Also, if stored flat, the pre-curvature can be lost to some degree over time as the stored material ages. Further, the degree of pre-curvature is set. Because rib bones vary so much in curvature, it would be desirable to be able to tune the degree of pre-curvature more easily so that curvatures can be tuned with an adaptive fit to each patient. Further, some bones such as rib bone may have all three kinds of curvature described above. It would be desirable to incorporate multiple kinds of curvature into the bone plates. Accordingly, the need exists for a bone plate that exhibits an adaptive fit to bones having multiple curvatures where the bone plates can be packaged efficiently and the degree of curvature can be more easily adapted and adjusted during surgery.

### SUMMARY OF THE DISCLOSURE

The present invention provides an adaptive fit, surgical bone plate system with fastener induced curvature. The bone plate system is useful to fixate injured bones. The bone plate system generally includes a bone plate including an array of fastener apertures and a plurality of fasteners used to secure the bone plate to bone tissue. The fastener apertures are undersized relative to the fasteners. The installation of fasteners into undersized fastener apertures in the bone plate induces increased curvature in the bone plates with respect to at least two of bending curvature, twisting curvature, and lateral curvature. In illustrative embodiments, the increased curvature includes at least increased bending curvature and increased twisting curvature.

In illustrative embodiments, the present invention uses fasteners that are oversized relative to the corresponding fastener apertures of the bone plate, and the fasteners are made from a harder material than the pad portions of a bone plate in which the apertures are formed. The forces that result from the volumetric displacement of bone plate material due to fastener insertion causes the bone plate to deform and contort to increase at least two of the bending, twisting, and/or lateral curvature of the bone plate. The deformation and contortion occur even if the fastener installed through the bone plate into open air, so that it is not merely the action of forcing conformation of the bone plate against the bone tissue that induces the adaptive fit. Bone plates may incorporate features such as ribs, scallops, windows, adjustment of thickness, initial native curvature, or the like to help induce complex geometry made from a combination of two or more of bending, twisting, and lateral curvature.

The induced curvature provides a new way to achieve similar functionality to pre-formed curvature or permanently bent and contoured curvature. In a simple and intuitive manner, installing fasteners in the fastener apertures allows patient specific curvature to be created in situ, helping to conform the plate to near anatomical geometry of the bone tissue, thus providing a plate form individually customized for the patient. The torque applied to the installed fasteners can be used to tune the degree of curvatures that are induced. The principles of the present invention allow planar bone plates to be fabricated that have an adaptive fit. The bone plate may have an initial, native geometry that can be contorted and deformed in a controllable manner using fastener insertion in order to more closely mirror the anatomical geometry of the underlying bone tissue.

In some embodiments, bone plates of the present invention have a native configuration that is planar with pre-formed lateral curvature. Fastener insertion induces increased bending and twisting curvature. This is very useful for fixation of bones with complex geometry, such as for example, rib bones. The ability of the bone plates to experience induced twisting curvature as well as induced bending curvature is provided in part by features incorporated into the bone plate that promote twisting when the oversized fasteners are inserted into the bone plate. In practical effect, due to the lateral curvature, bending the bone plate also twists the bone plate.

In one aspect, the present disclosure provides a surgical bone plate system useful to help fixate at least first and second underlying bone portions. The system includes a polymeric bone plate comprising at least one biocompatible polymer material. The polymeric bone plate comprises a native configuration and an induced multi-curved configuration. The induced multi-curved configuration includes increased curvature comprising at least two of increased bending curvature, increased twisting curvature, and/or increased lateral curvature relative to the native configuration. The surgical bone plate system further includes a 1×n aperture array of fastener apertures formed in the at least one biocompatible polymer material of the polymeric bone plate and extending along a length of the polymeric bone plate in a direction from a first bone plate end to a second bone plate end. The 1×n aperture array comprises n fastener apertures formed in the at least one biocompatible polymer material, and wherein n is 6 or more. The surgical bone plate system further includes a plurality of fasteners that are configured to be installed in the fastener apertures and into the at least first and second underlying bone portions in a manner effective to couple the polymeric bone plate to the at least first and second underlying bone portions. The fastener apertures are undersized relative to the fasteners, and the fasteners comprises a fastener material that is harder than the at least one biocompatible polymer material in which the fastener apertures are formed such that inserting the fasteners into the fastener apertures of the plurality of fastener apertures volumetrically displaces the at least one biocompatible polymer in a manner to induce the polymeric bone plate to transition from the native configuration to the multi-curved configuration.

In another aspect, the present invention provides a surgical bone plate system useful to help fixate at least first and second underlying bone portions, comprising:
a) a polymeric bone plate comprising at least one biocompatible polymer material, wherein the polymeric bone plate comprises a native configuration and an induced multi-curved configuration, and wherein the induced multi-curved configuration includes increased curvatures comprising at least two of increased bending curvature, increased twisting curvature, and/or increased lateral curvature relative to the native configuration;
b) a 1×n aperture array formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is in the range from 6 to 50, wherein each fastener aperture of the plurality of fastener apertures is provided at a region of the polymeric bone plate having an associated plate thickness, and wherein each fastener aperture of the plurality of fastener apertures has a plate aperture diameter; and
c) a plurality of fasteners that are configured to be installed in the fastener apertures and in the at least first and second underlying bone portions in a manner effective to couple the polymeric bone plate to the at least first and second underlying bone portions, wherein each fastener of the plurality of fasteners has a head diameter, wherein each fastener of the plurality of fasteners is oversized relative to a corresponding aperture of the plurality of fastener apertures, and wherein each fastener of the plurality of fasteners and a corresponding aperture of the plurality of fastener apertures have a volumetric displacement relationship that is selected from one or more ratios in the group consisting of:
   1) a ratio of volumetric displacement to plate thickness in the range from 0.55 to 1.2;
   2) a ratio of volumetric displacement to plate aperture diameter in the range from 0.45 to 1.2; and/or
   3) a ratio of volumetric displacement to fastener head diameter in the range from 0.30 to 1.00.

In another aspect, the present invention provides a bone plate system useful to help fixate at least first and second underlying bone portions, comprising:
a) a polymeric bone plate comprising at least one biocompatible polymer material, wherein the polymeric bone plate comprises lateral curvature in a native configuration;
b) a 1×n aperture array of fastener apertures formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is 6 or more; and
c) a plurality of metal fasteners that are configured to be installed in the fastener apertures and into the at least first and second underlying bone portions in a manner effective to couple the polymeric bone plate to the at least first and second underlying bone portions, wherein each metal fastener of the plurality of metal fasteners is oversize relative to a corresponding fastener aperture of the plurality of fastener apertures.

In another aspect, the present method comprises a method of making a bone plate system. The method includes steps comprising
a) providing a polymeric bone plate comprising at least one biocompatible polymer material, wherein the polymeric bone plate comprises a native configuration and an induced multi-curved configuration, and wherein the induced multi-curved configuration includes increased curvature comprising at least two of increased bending curvature, increased twisting curvature, and/or increased lateral curvature relative to the native configuration;
b) providing a 1×n aperture array of fastener apertures formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is 6 or more; and
c) providing a plurality of fasteners that are configured to be installed in the fastener apertures and into the at least first and second underlying bone portions in a manner effective to couple the polymeric bone plate to the at least first and second underlying bone portions; and

wherein the fastener apertures are undersized relative to the fasteners and wherein the fasteners comprises a fastener material that is harder than the at least one biocompatible polymer material in which the fastener apertures are formed such that inserting the fasteners into the fastener apertures of the plurality of fastener apertures volumetrically displaces the at least one biocompatible polymer in a manner to induce the polymeric bone plate to transition from the native configuration to the multi-curved configuration.

In another aspect, the present invention relates to a method of fixating at least two bone portions. The method comprises the steps of:
a) providing a bone plate system comprising at least one biocompatible polymer material, wherein the polymeric bone plate comprises a native configuration and an induced multi-curved configuration, and wherein the induced multi-curved configuration includes increased curvature comprising at least two of increased bending curvature, increased twisting curvature, and/or increased lateral curvature relative to the native configuration;
b) providing a 1×n aperture array of fastener apertures formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is 6 or more; and
c) providing a plurality of fasteners that are configured to be installed in the fastener apertures and into the at least first and second underlying bone portions in a manner effective to couple the polymeric bone plate to the at least first and second underlying bone portions; and

wherein the fastener apertures are undersized relative to the fasteners and wherein the fasteners comprises a fastener material that is harder than the at least one biocompatible polymer material in which the fastener apertures are formed such that inserting the fasteners into the fastener apertures of the plurality of fastener apertures volumetrically displaces the at least one biocompatible polymer in a manner to induce the polymeric bone plate to transition from the native configuration to the multi-curved configuration; and
b) using the bone plate system to fixate at least the first and second rib bone portions. As used herein:

The term “unitary” refer to a construction that is a single piece, although it may comprise elements that can be separately named, or to a construction that is formed from multiple pieces where multiple pieces are permanently joined (such as by welding, permanent adhesive, permanent fasteners, or methods which cannot be reversed non-destructively) to form a single piece construction; and

The term “or” is generally employed in its sense including “and/or” unless the content expressly states otherwise.

Each of the terms “have”, “having”, “contain”, “containing”, “characterized by”, “include”, “including”, “comprise”, “comprising” or the like is open-ended and does not exclude additional, unrecited elements, method steps, or other features.

The preceding summary of the present disclosure is not intended to describe each embodiment of the present invention. The details of one or more embodiments of the invention are also set forth in the description below. Other features, objects, and advantages of the invention will be apparent from the description and from the claims.

### BRIEF DESCRIPTION OF THE DRAWING

FIG. 1A is a top plan view of one embodiment of a bone plate according to the present invention having in-plane radius curvature in a native configuration.

FIG. 1B is a side view of the bone plate of FIG. 1A.

FIG. 2A is a generally side perspective view of the bone plate of FIG. 1A in which fasteners are inserted into the bone plate, wherein the inserted fasteners induce the bone plate to have increased out of plane radius curvature (bending) and increased longitudinal twist (twisting) curvature.

FIG. 2B is a generally top perspective view of the bone plate and fasteners of FIG. 2A showing the increased bending and twisting induced by insertion of the fasteners into the bone plate.

FIG. 2C is another generally side perspective view of the bone plate and fasteners of FIG. 2A showing the increased bending and twisting induced by insertion of fasteners.

FIG. 3 is a side-view showing first and second, generally identical bone plates according to the present invention, wherein one of the bone plates is in a native configuration and the other bone plate is in an induced multi-curved configuration resulting from fastener insertion.

FIG. 4A shows how a curve has a length and a height.

FIG. 4B shows how a chord and an associated sagitta having the same dimensions as the length and height of the curve of FIG. 4A define one associated circle have a particular radius, wherein the inverse of the radius is useful to define the curvature of the curve of FIG. 4A.

FIG. 4C schematically shows how the curvature of the bone plate of FIG. 1A may be determined in accordance with principles of FIGS. 4A and 4B.

FIG. 4D shows a graphical depiction for a method of calculating degrees of curvature.

FIG. 4E shows a graphical depiction for a method of calculating degrees of curvature for complex curves.

FIG. 5 is a side perspective view of one embodiment of a fastener according to the present invention.

FIGS. 6A-6F are schematic cross-sections of different bone plate segments according to the present invention, wherein each segment includes an illustrative embodiment of a fastener aperture profile useful in the practice of the present invention.

FIG. 7A is a schematic cross-section of a fastener and bone plate segment having a fastener aperture, wherein the fastener aperture is undersized relative to the fastener.

FIG. 7B is a schematic cross-section of the fastener and bone plate of FIG. 7B showing how the fastener threadably engages and volumetrically displaces bone plate material when the fastener is installed into the bone plate segment, wherein the volumetric displacement induces forces that cause curvature of the bone plate to increase.

FIG. 8A is a top view of an embodiment of a planar bone plate used in the surgical bone plate system of FIG. 8B, wherein the bone plate is in a native configuration.

FIG. 8B is a perspective view of a surgical bone plate system of the present invention including the bone plate of FIG. 8A and a plurality of fasteners inserted in the bone plate, wherein the bone plate is in a multi-curved configuration including increased bending curvature and increased twisting curvature.

FIG. 9A shows a top view of an alternative embodiment of a surgical bone plate of the present invention in a native configuration, wherein the native configuration includes lateral curvature, and wherein the surgical bone plate may be used in place of the bone plates shown in FIGS. 2A, 2B, 2C, 8A, and 8B.

FIG. 9B. is a perspective view of the bone plate of FIG. 9A.

FIG. 10A is a top (anterior) view of an alternative embodiment of a surgical bone plate of the present invention in a native configuration, wherein the native configuration includes lateral curvature, and wherein the surgical bone plate may be used in place of the bone plates shown in FIGS. 2A, 2B, 2C, 8A, and 8B.

FIG. 10B is a bottom (posterior) view of the surgical bone plate of FIG. 10A.

FIG. 10C is perspective view of the surgical bone plate of FIG. 10A.

FIG. 11A a top (anterior) view of an alternate embodiment of a surgical bone plate of the present invention in its native configuration.

FIG. 11B is a perspective view of the surgical bone plate of FIG. 11A.

### DETAILED DESCRIPTION

The present invention will now be further described with reference to the following illustrative embodiments. The embodiments of the present invention described below are not intended to be exhaustive or to limit the invention to the precise forms disclosed in the following detailed description. Rather a purpose of the embodiments chosen and described is so that the appreciation and understanding by others skilled in the art of the principles and practices of the present invention can be facilitated.

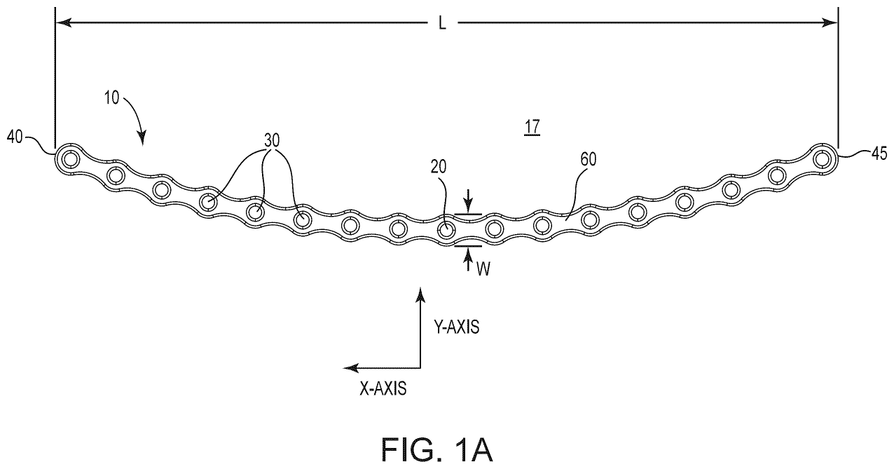

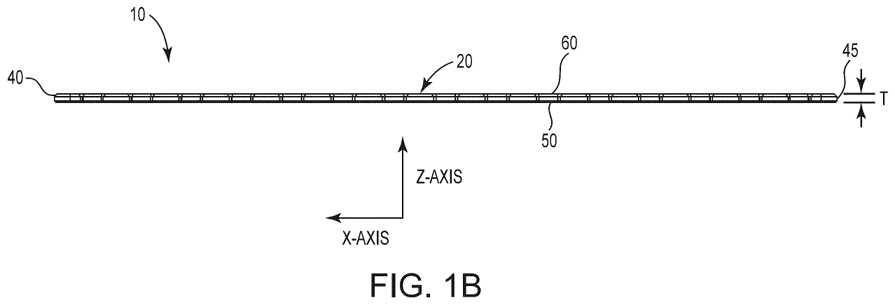

FIGS. 1A, 1B, 2A, 2B, 2C, and FIG. 5 shown an illustrative embodiment of a bone plate system 15 of the present invention. Bone plate system 15 is useful to help fixate at least first and second underlying bone portions (not shown), such as may occur when a bone such as a rib bone (not shown) has been broken or fractured. The bone plate system 15 includes a bone plate 10 and a plurality of fasteners 70.

As will be described below, the bone plate 10 and fasteners 70 are configured so that insertion of fasteners 70 into the bone plate 10 causes increased curvature of the bone plate with respect to two or more of bending curvature, twisting curvature, and lateral curvature, and preferably increased bending curvature and increased twisting curvature. This increased curvature helps to provide an adaptive fit of the bone plate 10 to the underlying bone tissue as the bone plate distorts as fasteners 70 are used to attach the bone plate 10 to the underlying rib bone portions. In practical effect, interactions among the fasteners 70 and bone plate 10 induce degrees of curvature into the bone plate 10 that mimic the functionality of providing a bone plate with pre-curvature or that results by permanently bending a bone plate into a curvature to match the anatomy of the underlying bone. This allows, if desired, bone plate 10 to be in an initial configuration, before fasteners 70 are installed, that is substantially planar. This allows for easy storage and packaging, as the induced curvature is not yet present until the fasteners 70 are installed. Installation of the fasteners then induces a conformation change by which the fit of the bone plate to the bone tissue can be adapted and customized for the patient.

Moreover, the degree of curvature of bone plate 10 can be tuned depending on how much torque is used to install the fasteners. For example, in illustrative embodiments, the degree of bending curvature of bone plate 10 can be adjusted from a range of 0 m⁻¹ to 20⁻¹, preferably 2⁻¹ to 15⁻¹, more preferably from 2⁻¹ to 12⁻¹ depending on the torque used to install each of the fasteners 70. Generally, using more torque tends to induce greater curvature, while using less torque tends to induce lesser curvature. The torque can be varied from fastener to fastener so that the degree of curvature can be tuned to greater and lesser degrees along the length of bone plate 10.

FIGS. 1A & 1B show top and side views, respectively, of surgical bone plate 10 according to the present invention in its native configuration. The native configuration refers to the configuration that the bone plate 10 naturally assumes when set down onto a flat surface 17 with posterior (bottom) surface 50 facing downward against the supporting surface and the anterior (top) surface 60 facing upward. In surgical use, the posterior surface 50 would be placed facing toward the underlying bone, while the anterior surface 60 would face upward away from the bone. In the native configuration, there are no fasteners 70 installed in the bone plate 10. Other than gravity and the supporting force of the flat surface acting on the bone plate 10, in the native configuration no other forces are acting on the bone plate 10 such as to stretch or twist or compress the bone plate 10 or to cause lesser or greater curvature than the bone plate 10 assumes on its own accord.

Bone plate 10 has at least one longitudinal twist inducing characteristic such that installation of fasteners 70 into the bone plate 10 will induce increased twisting curvature. As can be seen best in FIG. 1A, bone plate 10 is formed with an initial degree of in-plane radius, or lateral, curvature in the native configuration. The presence of this curvature helps to induce increased twisting curvature in bone plate 10 when fasteners 70 are installed in the bone plate 10. Any suitable curvature profile may be employed, for example, circular, elliptical, parabolic, logarithmic, or any other curve. The curvature profile may be symmetric or asymmetric. The bone plate may incorporate multiple curved sections (not shown) that meet at inflection points.

Bone plate 10 may be formed from a wide variety of materials. Preferably, bone plate 10 is formed from one or more biocompatible polymers. One or more biocompatible polymers are useful, as using these to form the bone plate 10, particularly in regions including fastener apertures 30, allows the fasteners 70 to volumetrically displace the bone plate material when the screws are inserted into the bone plate 10. In practical effect, the apertures 30 are undersized relative to the fasteners 70 so that the fasteners not only threadably engage the biocompatible polymeric material, but also forcibly push the material of the bone plate aside in order to allow the apertures 30 to increase sufficiently in size to allow the fasteners 70 to be inserted through the apertures 30 and screwed into the underlying bone tissue. These volumetric displacement forces are believed to be a key reason as to why insertion of the fasteners into the bone plate 10 induces increased curvature in the bone plate 10.

Examples of biocompatible polymer materials useful for forming bone plate 10 include one or more of PEEK (Polyether Ether Ketone), PLLA (Poly-L-Lactic Acid), PGA (Polyglycolic Acid), PVC (polyvinyl chloride), PE (polyethelene), PP (polypropylene), PLA (Polylactic Acid), PTFE (Polytetrafluoroethylene), PMMA (Polymethyl Methacrylate), silicone, PTMC (polytrimethylcarbonate), PVDF (Polyvinylidene Fluoride), UHMWPE (Ultra-High-Molecular-Weight Polyethylene) and combinations of these. PEEK is a preferred biocompatible polymer for use in bone plate 10.

Bone plates may be unitary articles or may be assemblies of multiple components. Bone plates may be manufactured by any suitable process, including injection molding, stamping, other molding process, machining, or the like.

Bone plate 10 comprises a plurality of fastener apertures 30. In a preferred mode of practice as shown, the apertures are arranged in a 1×n aperture array extending along a length of the bone plate in a direction from a first bone plate end 40 to a second bone plate end 45, where n is the number of apertures and n is at least 6 and preferably is in the range from 6 to 50. If n is below 6, the array of apertures could be ineffective at helping to induce twisting curvature when fasteners 70 are installed. For example, a prior art bone plate having a 1×4 array of holes and commercially sold as a bone plate in the VALKERIE fixation system commercially available from Able Medical Devices, Marquette, MI, does not noticeably undergo any induced twisting when fasteners are installed in the bone plate. The upper limit for n may be any desired number depending upon the lengths of the bone portions being fixated. For many instances, n may be in the range from 6 to 50, preferably 8 to 50, more preferably 8 to 40, or even 8 to 25 or even 9 to 25 fastener apertures 70. Illustrative embodiments of bone plates having n=11 or n=17, respectively, would be suitable for human rib bone fixation. For purposes of illustration, FIGS. 1A & 1B show an illustrative embodiment of bone plate 10 having seventeen fastener apertures 30 spaced between first bone plate end 40 and second bone plate end 45.

As shown in FIGS. 1A and 1B, bone plate 10 has a length L, a width W, and a thickness T. The length may vary over a wide range, depending on the length of the rib bone being treated and its injury (ies). Illustrative lengths may be in a range from 50 mm to 1000 mm. Illustrative widths may be in a range from 5 mm to 50 mm, and typical thicknesses between 1 mm to 10 mm.

FIGS. 1A & 1B also show X, Y, and Z axes of a Cartesian coordinate system useful to help describe curvatures of bone plate 10. The direction of the mutually perpendicular X, Y, and Z axes correspond to the length L, width W, and thickness T, respectively, of the bone plate 10 in its native configuration. In case a bone plate is not planar, for planar and nonplanar embodiments, the z axis is taken to be normal to the top surface 60 of bone plate 10 at center point 20 that is midway between first bone plate end 40 and second bone plate end 45 and midway across the width W of bone plate 10; the y axis is taken to be the axis along the width of the bone plate at the center point 20 perpendicular to the z-axis, and the x axis is taken to be the axis along the length of the bone plate 10 that is perpendicular to the y and z axes, and wherein the center point 20 is taken to be the intersection of the three, mutually perpendicular axes.

As used herein, “out of plane radius” curvature or “bending” curvature is defined as curvature around the y axis, “longitudinal twist” curvature or “twisting” is defined as curvature around the x axis such as in a helical manner, and “in-plane radius” curvature or “lateral” curvature is defined as curvature around the z axis. Consequently, FIGS. 1A and 1B depict an embodiment of a bone plate 10 which, in its depicted native configuration, has no out of plane radius curvature and no longitudinal twist curvature, but which has in-plane radius curvature. This embodiment of bone plate 10, thus, is planar.

The pre-formed, lateral curvature of bone plate 10 in the native configuration helps to induce increased twisting curvature when fasteners 70 are installed on bone plate 10. The installation of the fasteners 70 also induces increased bending curvature as well. The practical effect of this is that installation of the fasteners 70 in bone plate 10 and the underlying bone helps to induce curvature that, in practical effect, functions in the same manner as pre-formed lateral and bending curvature in bone plate 10.

In other embodiments, the native configuration of a bone plate may include any combination of out of plane radius, longitudinal twist, or in-plane radius curvatures. Installation of the fasteners 70 would cause at least two of these curvatures, preferably bending and twisting curvatures, to increase relative to the native configuration.

The out of plane radius, longitudinal twist, or in-plane radius curvatures of the native configuration may include or exclude curves that vary in degree of curvature over the length of the bone plate. The out of plane radius, longitudinal twist, or in-plane radius of the native configuration may include or may exclude curves that recurve over the length of the bone plate, such as S-curves provided that at least one section of an S-curve or other curve with multiple curved sections that meet at one or more inflection points includes 6 or more apertures in that section. As indicated above, 6 or more apertures is needed for installation of the fasteners 70 to induce twisting curvature.

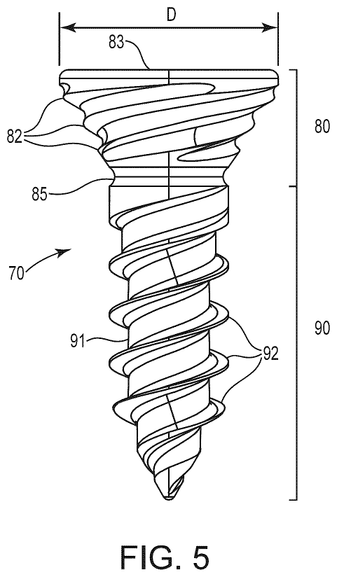

The bone plate system according to the present disclosure includes a plurality of fasteners 70 configured to be installed in the fastener apertures 30 of the bone plate 10 to couple the bone plate 10 to underlying portions (not shown) being fixated. An illustrative configuration of fasteners 70 is shown in FIG. 5. FIG. 5 shows that each fastener 70 includes a threaded and tapered head 80 and a threaded shaft 90. Preferably at least six fasteners 70 used in bone plate 10 have this configuration. Preferably, all of the fasteners 70 used in bone plate 10 have this configuration. This configuration has features that help to induce increased curvature in the bone plate 10.

As shown in the configuration of FIG. 5, fastener 70 includes a tapered and threaded head portion 80 and a threaded shaft portion 90. Tapered and threaded head portion 80 includes threads 82. Tapered and threaded head portion 80 includes functionality in which the threads 82 threadably engage the sidewall of a corresponding fastener aperture 30. Given that the fastener apertures 30 are undersized relative to the associated fastener 70, and given that fasteners 70 are made from a harder material (e.g., a metal material) that is harder than the polymeric bone plate 10, a fastener 70 volumetrically displaces adjacent material of bone plate 10 as the head portion 80 threadably engages the sidewall of the associated aperture 30. The volumetric displacement helps to induce at least increased bending and twisting curvature of the bone plate 10 in this embodiment. Consequently, the threads 82 are configured to facilitate this purpose.

FIG. 5 shows a view of one illustrative fastener 70 of the plurality of fasteners 70. In this preferred embodiment, head portion 80 is tapered so that head portion 80 is wider proximal to the top 83 and narrows in a direction toward the shaft portion 90. Thus, head portion has a diameter D at top 83 that is wider than the diameter of fastener 70 at juncture 85 between the head portion 80 and the shaft portion 90. The tapered shape of head portion 80 helps to induce increased curvature in bone plate 10. Because tapered head portion 80 is wider at the top 83 and narrows toward junction 85, the head portion 80 tends to exert larger volumetric displacement forces where head portion 80 is wider and lesser volumetric displacement forces where head portion 80 is narrower. This variation in the magnitude of the volumetric displacement forces is believed to help induce the increased bending and twisting curvatures in the bone plate 10.

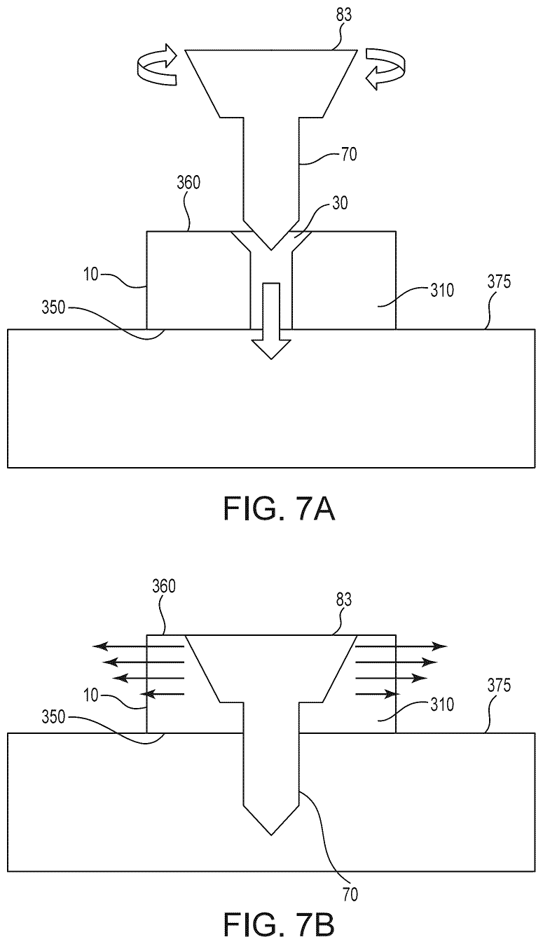

An advantageous feature of this displacement is that the degree of curvature can be adjusted by how much torque is used to install fasteners 70. If a fastener 70 is installed to a higher torque specification, the installation will induce the bone plate to increase curvature to a greater degree than if the fastener is installed to a lower torque specification. The torque varies at least in part due to the relationship between the tapered head portion 80 and the undersized, corresponding fastener aperture 30. Quite simply, it takes more torque to drive the wider top 83 deeper into the corresponding aperture 30, particularly in those embodiments in which the corresponding aperture also tapers from a direction from the opening of the aperture through which the fastener 70 is inserted toward the anterior side 50 of the bone plate. This will be described further below in connection with FIGS. 7A and 7B.

The top 83 of head portion 80 bears features (not visible) enabling engagement with a suitable installation and or removal tool, such as (without limitation) hex head features, Phillips head features, recessed square head features, star head features, and the like.

Shaft portion 90 includes shaft 91 and threads 92. Shaft portion 90 has a functionality that includes threadably engaging an underlying rib bone portion during installation and thereby helps bone plate 10 to hold the rib bone portion fixated after installation. Consequently, the profile of shaft 91 and the characteristics of threads 92 are adapted to that purpose.

Fasteners 70 may comprise any biocompatible material of sufficiently greater hardness than the polymer material of the bone plate in which apertures 30 are formed such that the fasteners 70 tend to volumetrically displace bone plate material upon insertion into the apertures 30 of bone plate 10 rather than the bone plate 10 predominantly compressing the fastener 70. Examples of suitable materials to use in fasteners 70 include one or more biocompatible polymers that are sufficiently harder than the biocompatible polymer(s) of the bone plate as well as one or metallic materials such as stainless steel, titanium or titanium alloys, cobalt-chromium alloys, ceramics, ceramic/metal composites, combinations of these, and the like. Titanium is one preferred material.

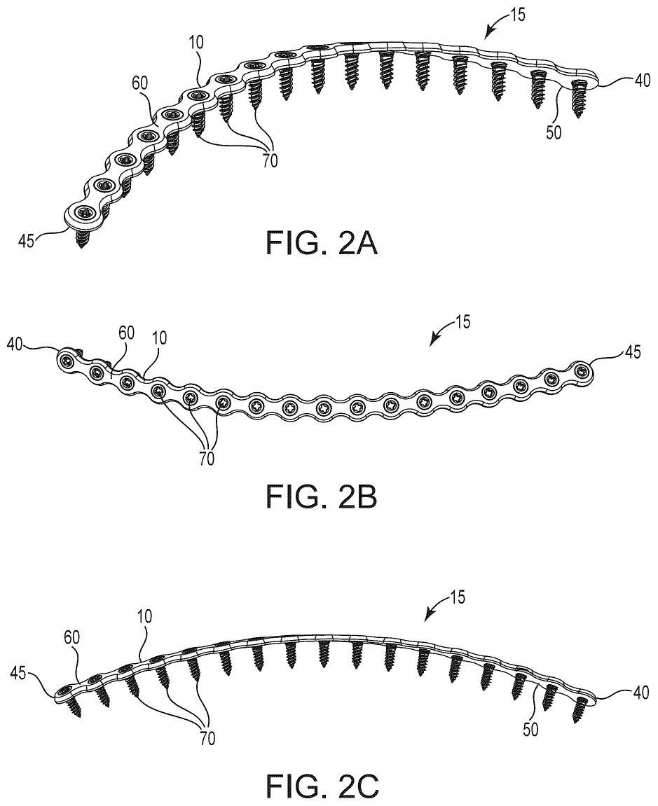

The bone plate 10 has a native configuration as shown in FIGS. 1A and 1B and an induced multi-curved configuration shown in FIGS. 2A, 2B, and 2C. The induced multi-curved configuration is characterized by increased out of plane curvature (also referred to herein as bending) and increased longitudinal twist (also referred to herein as twisting) relative to the native configuration. Such increased curvature results at least in part due to a combination of features including the threaded and tapered head portion 80 of fasteners 70 is tapered, the fastener apertures 30 in the bone plate 10 are undersized relative to the fasteners 70, and the fasteners 70 are made from a material harder than the material in which the apertures 30 are formed so that the installed fasteners 70 volumetrically displace bone plate material defining the apertures 30. As a result, threadably inserting the threaded heads of the fasteners 70 into the fastener apertures 30 volumetrically displaces portions of the polymeric material of the bone plate in a manner to induce the bone plate to transition from the native configuration to the multi-curved configuration.

FIGS. 2A, 2B, and 2C show how installation of fasteners 70 in fastener apertures 30 of bone plate 10 results in a change of configuration of bone plate 10 between the native configuration of FIGS. 1A and 1B to the induced multi-curved configuration of FIGS. 2A, 2B, and 2C as seen in bone plate system 15. The installation of fasteners 70 results in increased out of plane radius in the negative Z direction, that is, an increased out of plane radius curvature relative to the native configuration. The installation of fasteners 70 also results in increased longitudinal twist of bone plate 10 around the X axis, that is, an increased longitudinal twist relative to the native configuration. With reference to FIGS. 2A, 2B, and 2C, the longitudinal twist is evident in the manner by which posterior surface 50 becomes increasingly visible toward first bone plate end 40 and anterior surface 60 becomes increasingly visible toward second bone plate end 45.

It is noted that the induced configuration change results primarily due to the installation of the fasteners 70 into apertures 30 of bone plate 10. This change in configuration happens even if the fasteners 70 are inserted through the bone plate into open air as shown in FIG. 2A, 2B, or 2C or through bone plate 10 into underlying bone portions (not shown). This shows that a main factor contributing to the change in configuration is due to the manner by which the fasteners 70 engage and exert bending and twisting forces in the bone plate 10.

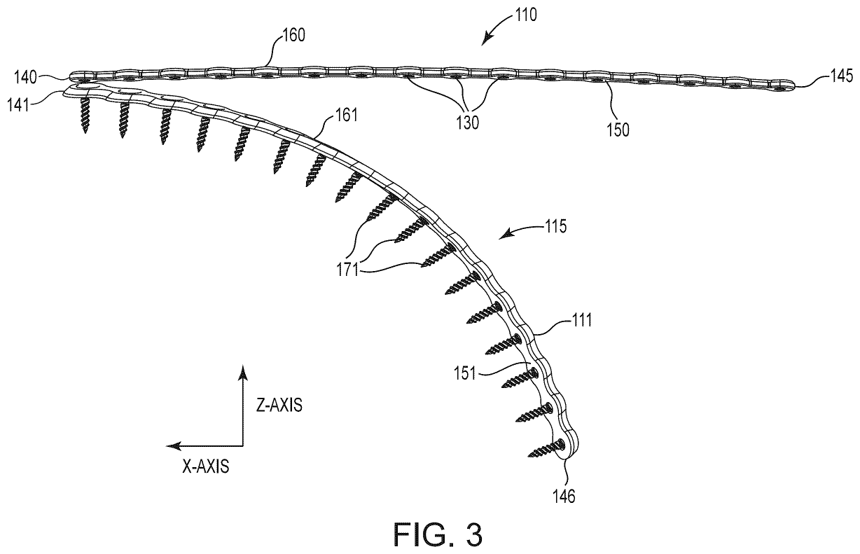

FIG. 3 demonstrates this in an “open air” experiment in which fasteners are driven into open air through one bone plate while an otherwise identical bone plate is left in its native configuration. FIG. 3 shows side views of two identical bone plate samples 110 and 111. Each of bone plate samples 110 and 111 has sixteen fastener apertures 130 arranged in a 1×16 array. Sample 111 differs from sample 110 in that Sample 111 includes 16 fasteners 171 inserted into the 16 apertures 130, respectively. Bone plate 110 is in the native configuration, having no fasteners inserted into its apertures 130. Bone plate 111 is in the induced multi-curved configuration bone plate 111 as a result of fasteners 171 being installed. Induced multi-curved configuration bone plate 111 together with fasteners 171 comprise bone plate system 115.

FIG. 3 expressly depicts X and Z axes. The Y axis is normal to the plane of the drawing and is perpendicular to the X and Z axes. Native configuration bone plate 110 comprises little or no out of plane radius curvature and little or no longitudinal twist. Bone plate 110 is substantially planar. Native configuration bone plate 110 comprises in-plane radius in the positive Y direction, into the page of the drawing. Bone plates 110, 111 comprise posterior surfaces 150, 151, designed to rest in whole or part against a rib bone after installation. Bone plates 110, 111 comprise anterior surfaces 160, 161 opposing posterior surfaces 150, 151. Bone plates 110, 111 comprise fastener apertures 130. The fastener apertures 130 in bone plate 111 are occupied by fasteners 171.

Bone plates 110, 111 differ only in that fasteners 171 are installed in induced multi-curved configuration bone plate 111 and not in native configuration bone plate 110. The installation of fasteners 171 in fastener apertures 130 causes the change of configuration of bone plate 111 from its native configuration to its induced multi-curved configuration. The installation of fasteners 171 results in increased out of plane radius curvature in the negative Z direction, that is, an increased degree of bending relative to the native configuration. The installation of fasteners 171 also results in increased longitudinal twist around the X axis, that is, an increased longitudinal twist relative to the native configuration. This is evident in the manner in which posterior surface 151 becomes increasingly visible toward second bone plate end 146 for multi-curved configuration bone plate 111, which is not true for native configuration bone plate 110.

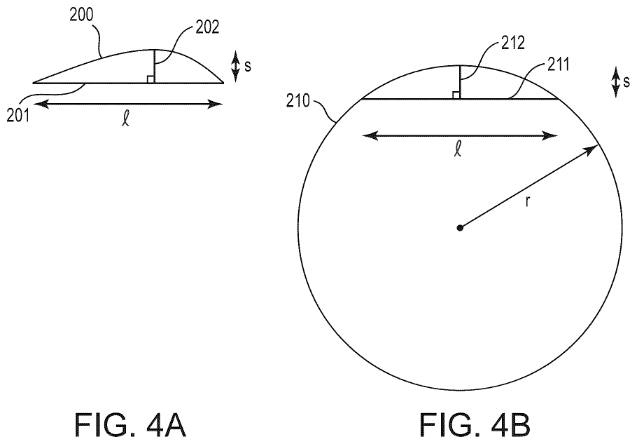

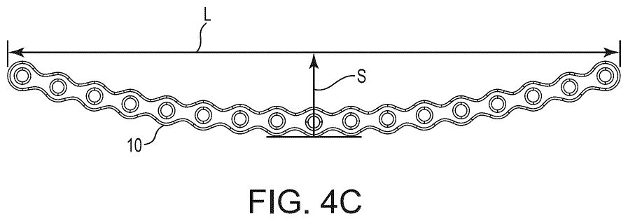

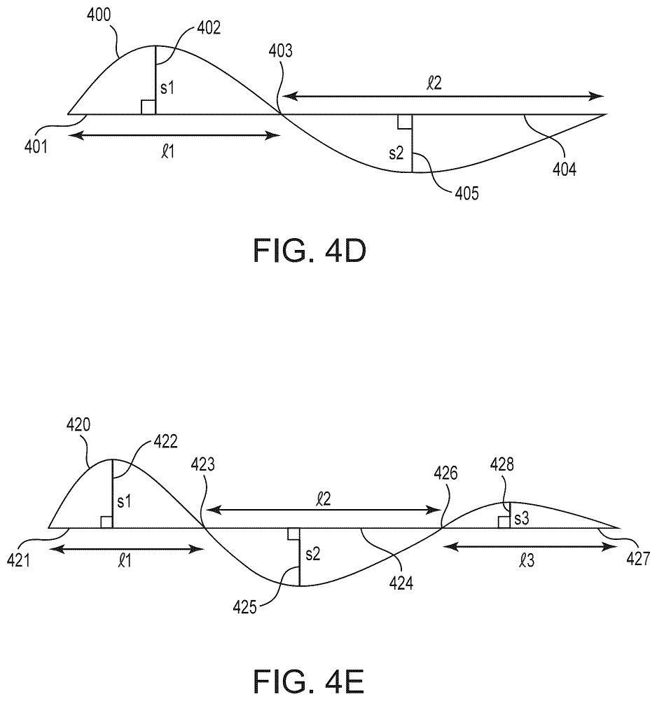

In the practice of the present invention, the degree of curvature of a circular or non-circular curve may be determined in terms of the inverse of the radius of an equivalent circle having a chord and a sagitta whose lengths, respectively, are the same as the baseline length of the curve section and height of the curve under examination. If you consider the line across the bottom of a curve to be a chord of a circle, and the height of the curve above the line to be the corresponding sagitta, then there is precisely one circle, and hence only one radius, that can have that chord length and associated sagitta. FIGS. 4A through 4E illustrate how the degree of curvature can be determined. With reference to FIG. 4A, a curve 200 has baseline of length 1 and a maximum height 202 from the baseline 201 to the curve having a length s. Referring to FIG. 4B, a equivalent circle 210 can be constructed having a chord 211 of length 1, equal to that of chord 201, and an associated sagitta 212 of length s, equal to that of line segment 202. The radius R of equivalent circle 210 may be used to express the degree of curvature of non-circular curve 200 as the value 1/r, or r⁻¹.

The radius r of the equivalent circle, and hence the degree of curvature given by 1/r, can be calculated by taking the length of baseline 201 to be the chord length 1 and the length of height 202 to be the sagitta s, according to the following formula:

$$r = \frac{{s}^{2} + \frac{1}{4} {l}^{2}}{2 s} = \frac{s}{2} + \frac{{l}^{2}}{8 s} .$$

where r is the radius of the circular arc, s is the sagitta length, and 1 (letter l) is the chord length. While any unit of length can be used, it is conventional in the bone plate field to calculate r in units of meters (m). The curvature is then given by the inverse of the radius, 1/r, in units of m⁻¹, inverse meters.

In the event that a curve is a composite of multiple curved sections connected at inflection points between the curved sections, curvature is determined for each curved section separately. FIG. 4D shows how to do this with an S curve 400 having two curved sections meeting at inflection point 403. The degree of curvature of the section of curve 400 to the left of inflection point 403 can be calculated using the above equation by using the length (L1) of base line 401 from the left end to the inflection point 403 as the chord length 1 and the length s1 of height 402 as the sagitta s in the above formula to calculate the radius. Similarly, the degree of curvature of the section of curve 400 to the right of inflection point 403 can be calculated using the above equation by using the length (L2) of base line 404 from the right end to the inflection point 403 as the chord length 1 and the length s2 of height 405 as the sagitta s in the above formula to calculate the radius.

If a curve were to be more complex such that one or more curved sections are connected by inflection points to each other or to the outer curved sections, FIG. 4E shows how the degree of curvature of each curve section of a curve 400 having three curved sections can be calculated. For example, between inflection points 423 and 426, base line 424 has a length L2 and a height 425 with length s2. L2 and S2 can be used in the above equation as the chord length 1 and sagitta length s to compute the radius r of the equivalent circle. From the left end to inflection point 423, base line 421 has a length L1 and a height 422 with length s1. L1 and S1 can be used in the above equation as the chord length 1 and sagitta length s to compute the radius r of the equivalent circle. From the right end to inflection point 426, base line 427 has a length L3 and a height 428 with length s3. L3 and S3 can be used in the above equation as the chord length 1 and sagitta length s to compute the radius r of the equivalent circle. For each of these calculations, the value 1/r is the degree of curvature for the corresponding curve section.

FIG. 4C shows how to apply the principles of an equivalent circle in order to determine the degree of curvature of bone plate 10 of FIGS. 1A, 1B, 2A, 2B, and 2C. Bone plate 10 has a length of L from end to end. A baseline of length L can be provided on the concave side of the lateral curvature so that the ends of the bone plate 10 in the native configuration is resting on the baseline. The curved bone plate has a maximum distance away from the baseline as shown by the line of length S that extends from the baseline to the distal edge of the bone plate. The length L and the length S can be taken as the chord length and sagitta length, respectively, in the formula above in order to determine the radius of an equivalent circle associated with such chord and sagitta. The inverse of the calculated radius, 1/r, is the degree of curvature.

In embodiments where the bone plate 10 of FIGS. 1A and 1B is formed with in-plane radius in its native configuration, The degree of this pre-formed in-plane radius curvature can vary over a wide range depending on how much twisting curvature is desired after the fasteners 70 are installed. A significant advantage of the present invention is that the degree of curvature can be adjusted at one or more points along the length of bone plate 10 by adjusting the installation torque of one or more fasteners 70.

In illustrative embodiments, the pre-formed, the in-plane radius curvature is typically in a range from greater than 1 m⁻¹ to 20 m⁻¹. In other embodiments, the in-plane radius curvature may be in a range from 1.5 m⁻¹ to 20 m⁻¹, from 2.0 m⁻¹ to 20 m⁻¹, from 3.0 m⁻¹ to 20 m⁻¹, from 4.0 m⁻¹ to 20 m⁻¹, from 1.0 m⁻¹ to 10 m⁻¹, from 2.0 m⁻¹ to 10 m⁻¹, from 3.0 m⁻¹ to 10 m⁻¹, or from 4.0 m⁻¹ to 10 m⁻¹.

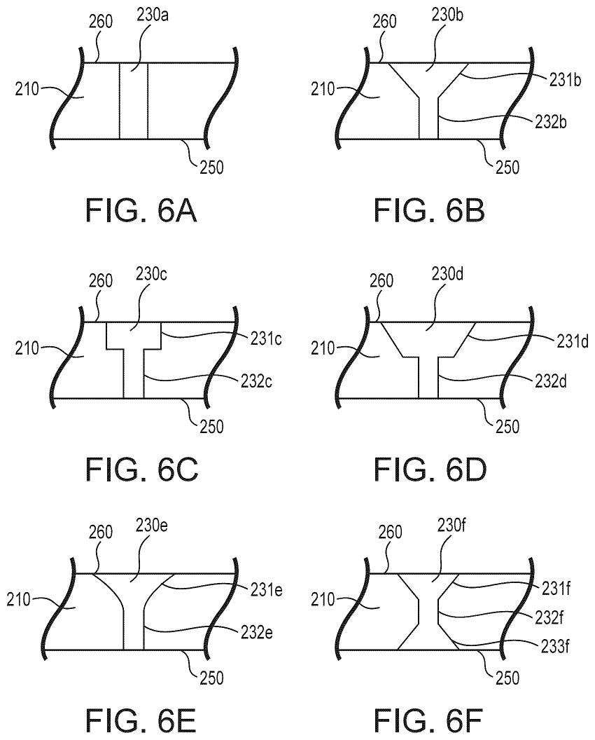

Fastener apertures useful in bone plates of the present invention, such as bone plates 10, 110, and 111 described above, and shown in the associated Figures, may have any suitable design. As examples, FIGS. 6A-6F schematically show segments of bone plates 210 in cross-section, each with an illustrative aperture configuration that may be used in the practice of the present invention. In each embodiment the wall(s) of the aperture are formed from the adjacent biocompatible polymer material(s) of the bone plate segment. Each bone plate 210 has a posterior surface 250 and an anterior surface 260. FIG. 6a depicts an embodiment wherein aperture 230a is a cylindrical through-hole of constant width. FIG. 6b depicts an embodiment wherein aperture 230b comprises a conical anterior chamfer segment 231b and a cylindrical shaft segment 232b. FIG. 6c depicts an embodiment wherein aperture 230c comprises a cylindrical anterior chamfer segment 231c and a cylindrical shaft segment 232c of lesser diameter. FIG. 6d depicts an embodiment wherein aperture 230d comprises a conical frustrum chamfer segment 231d and a cylindrical shaft segment 232d. FIG. 6e depicts an embodiment wherein aperture 230e comprises a curved-side “trumpet” chamfer segment 231e and a cylindrical shaft segment 232e. FIG. 6f depicts an embodiment wherein aperture 230f comprises a conical anterior chamfer segment 231f, a cylindrical shaft segment 232f, and a conical posterior chamfer segment 233f.

FIGS. 7A and 7B illustrate volumetric displacement of bone plate material 310 that occurs with installation of fastener 70 of FIGS. 2A, 2B, 2C and FIG. 5 in fastener aperture 30 formed in bone plate material 310 of bone plate 10 shown in FIGS. 1A, 1B, 2A, 2B, and 2C. FIGS. 7A and 7B illustrate a principle of the present invention by which at least a portion of the fastener aperture 30 is undersized relative to the girth of the corresponding fastener 70 to be installed. An aspect of the practice of the present invention is that the fastener 70 is made from a harder material than the bone plate material 310 so that insertion of fastener 70 causes fastener 70 not only to threadably engage and attach to the bone plate material 310, but to actually volumetrically displace bone plate material 310 due to the aperture 30 being undersized relative to the oversized fastener 70. This principle is beneficially incorporated into the practice of the present invention with respect to any of FIG. 1A through FIG. 6F.

The mismatch in size between fastener 70 and aperture 30 is best seen in FIG. 7A. In FIG. 7A, fastener 70 is aligned with aperture 30 but has not yet been installed. As an option, a suitable fastener guide can be used to help hold a pre-loaded fastener in alignment with a corresponding aperture. The guide is coupled to the bone plate in a manner effective to provide such alignment and then to help guide insertion of the fastener into the bone plate and underlying bone tissue. An example of such a guide is commercially available from Able Medical Devices, Marquette, MI, as an accessory used with the VALKYRIE™ Thoracic Fixation System.

As a result, installation of the oversized fastener 70 results in volumetrically displacement of portions of the polymeric material of the bone plate material 310 adjacent to aperture 30. As a result of this displacement as enough fasteners are installed along at least a portion of the bone plate the bone plate is induced to transition from the native configuration to the multi-curved configuration. The increased curvature results due to the forces exerted by the fastener 70 against the adjacent bone plate material 310.

FIG. 7B shows fastener 70 installed in bone plate material 310 and an underlying bone 375. FIG. 7B schematically illustrates displacement forces that the tapered head of fastener 70 exerts against the adjacent bone plate material 310. These forces are shown by the arrows projecting outward from the fastener 70 into the surrounding bone plate material 310. The magnitude of the exerted forces is shown schematically by the length of the arrows. Longer arrows correlate to great force exerted by the tapered head against the volumetrically displaced, adjacence bone plate material 310. Due to the tapered shape of the head of fastener 70, it can be seen that displacement forces are greater toward anterior surface 60, and lesser toward posterior surface 50. This force profile gradient, with large forces near the anterior surface 60, in combination with the pre-formed lateral curvature of the bone plate, induces increased bending and twisting curvature of the bone plate as fasteners are installed along the length of the bone plate. In some embodiments, volumetric displacement increases monotonically from the posterior surface to the anterior surface.

In some embodiments, the degree of increase in out of plane curvature and increase in longitudinal twist induced in the bone plate by installation of the fasteners 70 is dependent on the amount of torque applied to the fasteners 70 during installation. Fasteners may be installed sequentially along the length of the bone plate during installation. In one preferred embodiment, fasteners are installed sequentially and the amount of torque applied to the fasteners 70 is adjusted, sometimes tuning the installation of earlier installed fasteners if desired, to induce the desired amount of out of plane curvature and longitudinal twist, providing an improved fit of the bone plate to the underlying bone.

The relationship among fastener dimensions, bone plate dimensions, and aperture dimensions with respect to any of the fasteners, bone plates, and apertures discussed above in any of FIGS. 1A through 7B may be used to help provide desired curvature characteristics. Relating volume displacement to the relationships among the fasteners, bone plate, and apertures is very useful for creating favorable curvature inducing characteristics in the practice of the present invention.

Volume displacement is given by the expression V=F−A, wherein: V is the volume displacement, F is the volume of the fastener installed in the bone plate between the anterior and posterior surfaces of the bone plate, and A is the volume of the corresponding aperture in its native configuration prior to installation of the fastener. As a general principle, displacement forces, and hence the increases in curvature, are greater as the volume displacement increases.

For example, the volumetric displacement of bone plate system 15 of FIGS. 2A, 2B, and 2C may be selected from a wide range of values that are effective to induce increased curvature and twisting when fasteners 70 are installed. In illustrative embodiments, the volumetric displacement is between 1.10 mm³ and 2.4 mm³, between 1.45 mm³ and 2.0 mm³, between 1.50 mm³ and 2.0 mm³, between 1.55 mm³ and 2.0 mm³, or between 1.60 mm³ and 1.8 mm³.

In the practice of the present invention, it is preferred that a bone plate system of the present invention, such as shown in any of FIGS. 2A to 2C herein, satisfies one or more, preferably two or more, and preferably all three of the following relationships.

As one relationship, it is preferably that the ratio of the volumetric displacement in mm³ to the bone plate thickness in mm is selected to help promote desired bending and twisting of bone plate 10 when fasteners 70 are installed. The plate thickness is given by the height of the corresponding aperture 30 from the anterior surface 50 to the posterior surface 60. Generally, induced curvature increases as this ratio increases. In some embodiments, the ratio of volumetric displacement to bone plate thickness is between 0.30 mm² and 1.2 mm², between 0.55 mm² and 1.2 mm², between 0.60 mm² and 1.2 mm², between 0.65 mm² and 1.2 mm², or between 0.65 mm² and 80 mm².

As a second relationship, it is preferable that the ratio of the volumetric displacement to the plate aperture diameter is selected to help promote desired bending and twisting of bone plate 10 when fasteners 70 are installed. The plate aperture diameter is the largest diameter of the corresponding aperture 30 in the native configuration that later is threadably engaged and volumetrically displaced by the corresponding tapered and threaded head portion 80 (See FIG. 5). In various embodiments, the ratio of volumetric displacement to plate aperture diameter is between 0.20 mm² and 1.2 mm², between 0.45 mm² and 1.2 mm², between 0.45 mm² and 0.75 mm², or between 0.45 mm² and 0.65 mm².

As a third relationship, it is preferable if the ratio of the volumetric displacement to the fastener head diameter is selected to help promote desired bending and twisting of bone plate 10 when fasteners 70 are installed. The fastener head diameter is the largest diameter of the tapered and threaded head portion 80 (see FIG. 5) that threadably engages and volumetrically displaces adjacent bone plate material when installed in the bone plate 10. In various embodiments, the ratio of volumetric displacement to fastener head diameter is between 0.15 mm² and 1.0 mm², between 0.30 mm² and 0.6 mm², between 0.32 mm² and 0.5 mm², or between 0.32 mm² and 0.45 mm².

In one mode of practice, the ratio of volumetric displacement to bone plate thickness is between 0.65 mm² and 80 mm², the ratio of volumetric displacement to plate aperture diameter is between 0.45 mm² and 0.65 mm², and the ratio of volumetric displacement to fastener head diameter is between 0.32 mm² and 0.45 mm². In an illustrative embodiment, the ratio of volumetric displacement to bone plate thickness is 0.72 mm², the ratio of volumetric displacement to plate aperture diameter is 0.54 mm², and the ratio of volumetric displacement to fastener head diameter is 0.36 mm².

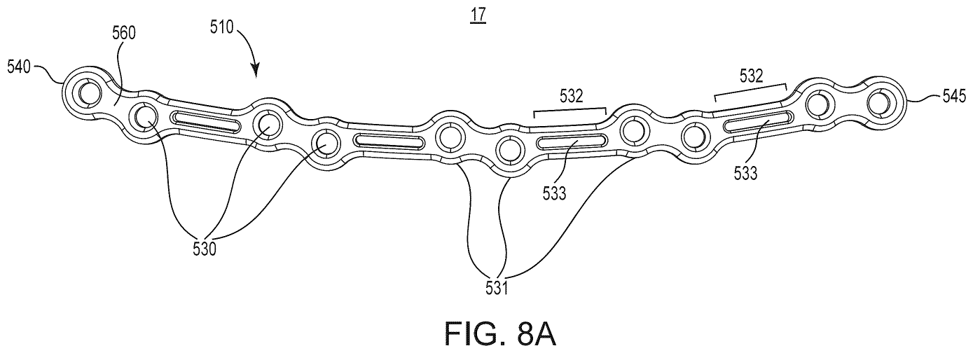

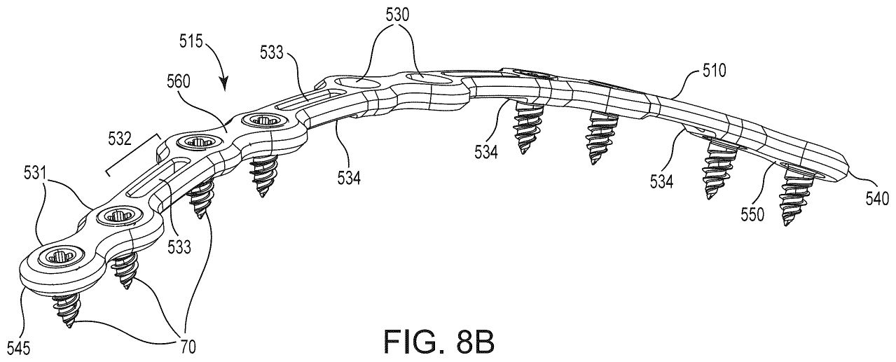

FIGS. 8A and 8B show a further embodiment of a surgical bone plate system 515 of the present invention useful to help fixate at least first and second underlying bone portions (not shown), such as may occur when a bone (not shown) has been broken or fractured. The surgical bone plate system 515 includes a surgical bone plate 510 and a plurality of fasteners which for purposes of illustration are the fasteners 70 of FIGS. 2A, 2B, 2C, and 5.

The bone plate 510 and fasteners 70 are configured so that insertion of fasteners 70 into the bone plate 510 causes increased curvature of the bone plate 510 with respect to at least two of bending, twisting, and lateral curvature, preferably including at least increased bending curvature and increased twisting curvature. This increased curvature helps to adapt the geometry of the bone plate 510 to the curved geometry of the underlying bone tissue. This allows, if desired, bone plate 510 to be in an initial configuration before fasteners 70 are installed that is substantially planar. This allows for easy storage and packaging, as the induced curvature is not yet present until the fasteners 70 are installed. The geometry of the bone plate 510 can them be adapted and customized to the geometry of the underlying bone tissue when the fasteners 70 are installed.

Moreover, the degree of curvature of bone plate 510 can be tuned depending on how much torque is used to install the fasteners 70. For example, in illustrative embodiments, the degree of bending curvature of bone plate 510 can be adjusted from a range of 0 m⁻¹ to 20 m⁻¹, preferably 2 m⁻¹ to 15 m⁻¹, more preferably from 2 m⁻¹ to 12 m⁻¹ depending on the torque used to install each of the fasteners 70. Generally, using more torque tends to induce greater curvature, while using less torque tends to induce lesser curvature. The torque can be varied from fastener to fastener so that the degree of curvature can be tuned to greater and lesser degrees along the length of bone plate 510.

FIG. 8A shows a top view of bone plate 510 according to the present invention in its native configuration. The native configuration refers to the configuration that the bone plate 510 naturally assumes when set down onto a flat surface 17 with posterior (bottom) surface 550 facing downward against the supporting surface and the anterior (top) surface 560 facing upward. In surgical use, the posterior surface 550 would be placed facing toward the underlying bone, while the anterior surface 560 would face upward away from the bone. In the native configuration, there are no fasteners installed in the bone plate 510. Other than gravity and the supporting force of the flat surface acting on the bone plate 510, no other forces are acting on the bone plate 510 such as to stretch or compress the bone plate 510 or to cause lesser or greater curvature than the bone plate 510 assumes on its own accord.

Bone plate 510 has at least one longitudinal twist inducing characteristic such that installation of fasteners, such as fasteners 70 of FIGS. 2A-C, 3, and 5, into the bone plate 510 will induce increased twisting curvature as well as increased bending curvature. As can be seen best in FIG. 8A, bone plate 510 is formed with an initial degree of in-plane radius, or lateral, curvature in the native configuration. The presence of this curvature helps to induce increased twisting curvature in bone plate 510 when fasteners 70 are installed in the bone plate 510. Any suitable curvature profile may be used for this pre-formed lateral curvature, for example, circular, elliptical, parabolic, logarithmic, or any other curve. The curvature profile may be symmetric or asymmetric. The bone plate may incorporate multiple curved sections (not shown) that meet at inflection points.

The degree of pre-formed lateral curvature may vary over a wide range. In some embodiments, the degree of pre-formed lateral curvature is in the range from 0.5 m⁻¹ to 12 m⁻¹, preferably 0.5 m⁻¹ to 8 m⁻¹, or even 1 m⁻¹ m to 8 m⁻¹.

Bone plate 510 may be formed from a wide variety of materials. Preferably, bone plate 510 is formed from one or more biocompatible polymers. One or more biocompatible polymers are useful, as using these to form the bone plate 510, particularly in regions including fastener apertures 530, allows the fasteners 70 to volumetrically displace the bone plate material when the screws are inserted into the bone plate 510. In practical effect, the apertures 530 are undersized relative to the fasteners 70 so that the fasteners not only threadably engage the biocompatible polymeric material, but also forcibly push the material aside in order to allow the apertures 530 to increase sufficiently in size to allow the fasteners 70 to be inserted through the apertures 530 and screwed into the underlying bone tissue. These volumetric displacement forces are believed to be a key reason as to why insertion of the fasteners into the bone plate 510 induces increased bending and twisting curvature in the bone plate 510.

Examples of biocompatible polymer materials useful for forming bone plate 510 include one or more of PEEK (Polyether Ether Ketone), PLLA (Poly-L-Lactic Acid), PGA (Polyglycolic Acid), PVC (polyvinyl chloride), PE (polyethelene), PP (polypropylene), PLA (Polylactic Acid), PTFE (Polytetrafluoroethylene), PMMA (Polymethyl Methacrylate), silicone, PTMC (polytrimethylcarbonate), PVDF (Polyvinylidene Fluoride), UHMWPE (Ultra-High-Molecular-Weight Polyethylene) and combinations of these. PEEK is a preferred biocompatible polymer for use in bone plate 510.

Bone plate 510 comprises a plurality of fastener apertures 530. In a preferred mode of practice as shown, the apertures are arranged in a 1×n aperture array extending along a length of the bone plate in a direction from a first bone plate end 540 to a second bone plate end 545, where n is the number of apertures and n is at least 6 and preferably is in the range from 6 to 50. If n is below 6, the array of apertures could be ineffective at helping to induce twisting curvature when fasteners 70 are installed. For many instances, n may be in the range from 6 to 50, preferably 8 to 50, more preferably 8 to 40, or even 8 to 25 or even 9 to 25 fastener apertures 70. For purposes of illustration, FIGS. 8A & B show an illustrative embodiment of bone plate 510 having ten fastener apertures 530 spaced between first bone plate end 540 and second bone plate end 545.

The length of bone plate 510 may vary over a wide range, depending on the length of the bone being treated and its injury (ies). Illustrative lengths may be in a range from 50 mm to 1000 mm. Illustrative widths may be in a range from 5 mm to 50 mm, and typical thicknesses between 1.0 mm to 10 mm.

FIGS. 8A and 8B depict an embodiment of a bone plate 510 which, in its depicted native configuration, has no out of plane radius curvature and no longitudinal twist curvature, but which has in-plane radius curvature. This embodiment of bone plate 510, thus, is planar. The pre-formed, lateral curvature of bone plate 510 helps to induce increased twisting curvature when fasteners 70 are installed on bone plate 510. The installation of the fasteners 70 also induces increased bending curvature as well. The practical effect of this is that installation of the fasteners 70 in bone plate 510 and the underlying bone helps to induce curvature that, in practical effect, functions as pre-formed lateral and bending curvature in bone plate 510.

The bone plate system according to the present disclosure includes a plurality of fasteners, such as fasteners 70 of FIGS. 2A-C, 3, and 5, configured to be installed in the fastener apertures 530 of the bone plate 510 to couple the bone plate 510 to underlying bone portions (not shown) being fixated. An illustrative configuration of fasteners 70 is shown in FIG. 5 and discussed above. In FIG. 8B, eight fasteners 70 are installed in eight of the ten available fastener apertures 530.

The bone plate 510 has a native configuration and an induced multi-curved configuration shown in FIG. 8B. The induced multi-curved configuration is characterized by increased out of plane curvature (also referred to herein as bending) and increased longitudinal twist (also referred to herein as twisting) relative to the native configuration. Such increased curvature results at least in part due to a combination of features including the threaded and tapered head portion 80 of fasteners 70 is tapered, the fastener apertures 530 in the bone plate 510 are undersized relative to the fasteners 70, and the fasteners 70 are made from a material harder than the material in which the apertures 530 are formed so that the installed fasteners 70 volumetrically displace bone plate material defining the apertures 530. As a result, threadably inserting the threaded heads 80 of the fasteners into fastener apertures 530 volumetrically displaces portions of the polymeric material of the bone plate in a manner to induce the bone plate to transition from the native configuration to the multi-curved configuration.

Additional features of bone plate 510 increase the ability of bone plate 510 to experience induced curvature changes when the oversized fasteners 70 are inserted into bone plate 510. Bone plate 510 comprises a plurality of fastener pads 531 deployed in pairs of pads 531 along the length of bone plate 510. Fastener apertures 520 are formed in pairs in each pair fastener pad 531 as well. The pairs of connected pads 531 are further connected to other pad pairs by beam sections 532. Pairs of fastener pads 531 (and hence pairs of fastener apertures 530) are deployed diagonally across the width of bone plate 510 and hence are diagonally connected by beam sections 532 along the length of bone plate 510. This diagonal geometry increases the degree to which volumetric displacement of the material of bone plate 510 induces curvature, particularly with respect to inducing increased twisting curvature.

Additional features of beam sections 532 also help to make it easier to induce increased curvatures in bone plate 510 when fasteners 70 are installed. These additional strategies reduce the amount of material in plate regions where induced curvatures will occur, thus making it easier to induces curvatures in beam sections 532. Beam sections 532 include windows 533, which are elongated. These windows also reduce the amount of material in beam sections 532. Windows 533 pass through beam sections 532 from anterior side 560 to posterior side 550. Additionally, beam sections 532 include scallops 534 in posterior side 550, wherein further material and the edge thickness of the beam sections 532 are reduced.

FIG. 8B shows how installation of fasteners 70 in fastener apertures 530 of bone plate 510 results in a change of configuration of bone plate 510 between the native configuration of FIG. 8A to the induced multi-curved configuration of FIG. 8B as seen in bone plate system 515. The installation of fasteners 70 results in increased out of plane radius in the negative Z direction, that is, an increased out of plane radius curvature relative to the native configuration. The installation of fasteners 70 also results in increased longitudinal twist of bone plate 510 around the X axis, that is, an increased longitudinal twist relative to the native configuration. With reference to FIGS. 8B, the longitudinal twist is evident in the manner by which posterior surface 550 becomes increasingly visible toward first bone plate end 540 and anterior surface 560 becomes increasingly visible toward second bone plate end 545.

It is noted that the induced configuration change results primarily due to the installation of the fasteners 70 into apertures 530 of bone plate 510. This change in configuration happens even if the fasteners 70 are inserted through the bone plate into open air as shown in FIG. 8B or through bone plate 510 into underlying bone portions (not shown). This shows that a main factor contributing to the change in configuration is due to the manner by which the fasteners 70 engage and exert bending and twisting forces in the bone plate 510.

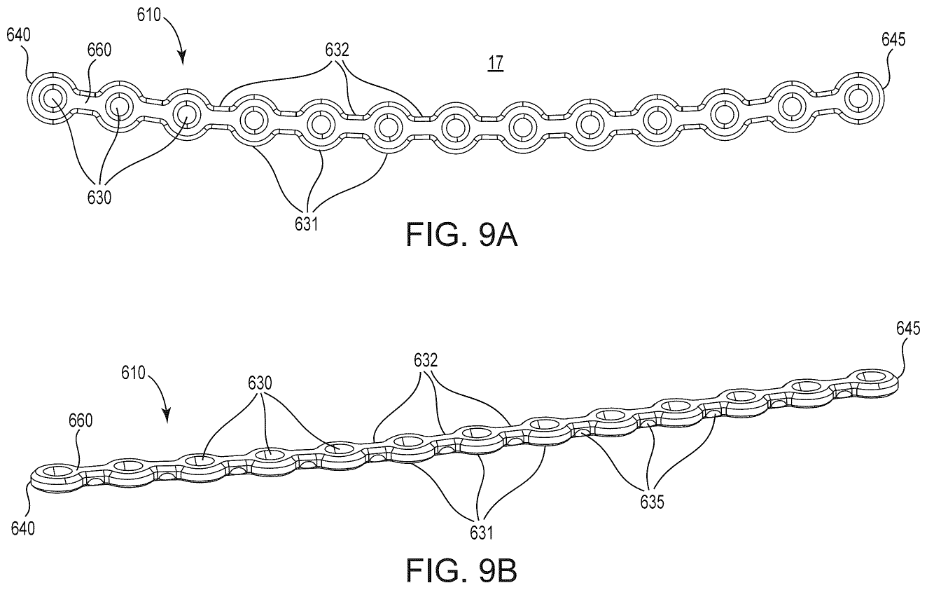

FIGS. 9A & 9B show a further embodiment of a surgical bone plate 610 of the present invention useful to help fixate at least first and second underlying bone portions (not shown), such as may occur when a bone (not shown) has been broken or fractured. A corresponding surgical bone plate system includes surgical bone plate 610 and a plurality of fasteners such as fasteners 70 of FIGS. 2A, 2B, 2C, and 5.

The bone plate 610 and fasteners such as fasteners 70 of FIGS. 2A-C, and 5 are configured so that insertion of fasteners into the bone plate 610 causes increased curvature of the bone plate with respect to at least two of bending, twisting, and lateral curvature, preferably including at least increased bending curvature and increased twisting curvature. This increased curvature helps to conform the bone plate to the geometry of the underlying bone tissue. This allows, if desired, bone plate 610 to be in an initial configuration before fasteners 70 are installed that is substantially planar. This allows for easy storage and packaging, as the induced curvature is not yet present until the fasteners are installed. The geometry of the bone plate can them be adapted and customized to the geometry of the underlying bone tissue when the fasteners are installed.

Moreover, the degree of curvature of bone plate 610 can be tuned depending on how much torque is used to install the fasteners. For example, in illustrative embodiments, the degree of bending curvature of bone plate 610 can be adjusted from a range of 0 m⁻¹ to 20 m⁻¹, preferably 2 m⁻¹ to 15 m⁻¹, more preferably from 2 m⁻¹ to 12 m⁻¹ depending on the torque used to install each of the fasteners. Generally, using more torque tends to induce greater curvature, while using less torque tends to induce lesser curvature. The torque can be varied from fastener to fastener so that the degree of curvature can be tuned to greater and lesser degrees along the length of bone plate 610.

FIG. 9A shows a top view of bone plate 610 according to the present invention in its native configuration. The native configuration refers to the configuration that the bone plate 610 naturally assumes when set down onto a flat surface 17 with posterior (bottom) surface (not visible) facing downward against the supporting surface and the anterior (top) surface 660 facing upward. In surgical use, the posterior surface would be placed facing toward the underlying bone, while the anterior surface 660 would face upward away from the bone. In the native configuration, there are no fasteners installed in the bone plate 610. Other than gravity and the supporting force of the flat surface acting on the bone plate 610, no other forces are acting on the bone plate 610 such as to stretch or compress the bone plate 610 or to cause lesser or greater curvature than the bone plate 610 assumes on its own accord.

Bone plate 610 has at least one longitudinal twist inducing characteristic such that installation of fasteners, such as fasteners 70 of FIGS. 2A-C, and 5, into the bone plate 610 will induce increased twisting curvature as well as increased bending curvature. As can be seen best in FIG. 9A, bone plate 610 is formed with an initial degree of in-plane radius, or lateral, curvature in the native configuration. The presence of this curvature helps to induce increased twisting curvature in bone plate 610 when fasteners are installed in the bone plate 610. Any suitable curvature profile may be employed, for example, circular, elliptical, parabolic, logarithmic, or any other curve. The curvature profile may be symmetric or asymmetric. The bone plate may incorporate multiple curved sections (not shown) that meet at inflection points.

The degree of pre-formed lateral curvature may vary over a wide range. In some embodiments, the degree of pre-formed lateral curvature is in the range from 0.5 m⁻¹ to 12 m⁻¹, preferably 0.5 m⁻¹ to 8 m⁻¹, or even 1 m⁻¹ m to 8 m⁻¹.

Bone plate 610 may be formed from a wide variety of materials. Preferably, bone plate 610 is formed from one or more biocompatible polymers. One or more biocompatible polymers are useful, as using these to form the bone plate 610, particularly in regions including fastener apertures 630, allows the fasteners to volumetrically displace the bone plate material when the screws are inserted into the bone plate 610. In practical effect, the apertures 630 are undersized relative to the fasteners so that the fasteners not only threadably engage the biocompatible polymeric material, but also forcibly push the material aside in order to allow the apertures 630 to increase sufficiently in size to allow the fasteners to be inserted through the apertures 630 and screwed into the underlying bone tissue. These volumetric displacement forces are believed to be a key reason as to why insertion of the fasteners into the bone plate 610 induces increased bending and twisting curvature in the bone plate 610.

Examples of biocompatible polymer materials useful for forming bone plate 610 include one or more of PEEK (Polyether Ether Ketone), PLLA (Poly-L-Lactic Acid), PGA (Polyglycolic Acid), PVC (polyvinyl chloride), PE (polyethelene), PP (polypropylene), PLA (Polylactic Acid), PTFE (Polytetrafluoroethylene), PMMA (Polymethyl Methacrylate), silicone, PTMC (polytrimethylcarbonate), PVDF (Polyvinylidene Fluoride), UHMWPE (Ultra-High-Molecular-Weight Polyethylene) and combinations of these. PEEK is a preferred biocompatible polymer for use in bone plate 610.

Bone plate 610 comprises a plurality of fastener apertures 630. In a preferred mode of practice as shown, the apertures are arranged in a 1×n aperture array extending along a length of the bone plate in a direction from a first bone plate end 640 to a second bone plate end 645, where n is the number of apertures and n is at least 6 and preferably is in the range from 6 to 50. If n is below 6, the array of apertures could be ineffective at helping to induce twisting curvature when fasteners are installed. For many instances, n may be in the range from 6 to 50, preferably 8 to 50, more preferably 8 to 40, or even 8 to 25 or even 9 to 25 fastener apertures. For purposes of illustration, FIGS. 9A & B show an illustrative embodiment of bone plate 610 having thirteen fastener apertures 630 spaced between first bone plate end 640 and second bone plate end 645.

The length of bone plate 610 may vary over a wide range, depending on the length of the bone being treated and its injury (ies). Illustrative lengths may be in a range from 50 mm to 1000 mm. Illustrative widths may be in a range from 5 mm to 50 mm, and typical thicknesses between 1 mm to 10 mm.

FIGS. 9A and 9B depict an embodiment of a bone plate 610 which, in its depicted native configuration, has no out of plane radius curvature and no longitudinal twist curvature, but which has in-plane radius curvature. This embodiment of bone plate 610, thus, is planar. The pre-formed, lateral curvature of bone plate 610 helps to induce increased twisting curvature when fasteners are installed on bone plate 610. The installation of the fasteners also induces increased bending curvature as well. The practical effect of this is that installation of the fasteners in bone plate 610 and the underlying bone helps to induce curvature that, in practical effect, functions as pre-formed lateral and bending curvature in bone plate 610 or curvature that results by permanently bending a bone plate to achieve an anatomical fit.

The bone plate system according to the present disclosure includes a plurality of fasteners, such as fasteners 70 of FIGS. 2A-C, 3, and 5, configured to be installed in the fastener apertures 630 of the bone plate 610 to couple the bone plate 610 to underlying bone portions (not shown) being fixated. An illustrative configuration of fasteners 70 is shown in FIG. 5 and discussed above.

The bone plate 610 has a native configuration and an induced multi-curved configuration (not shown). The induced multi-curved configuration is characterized by increased out of plane curvature (also referred to herein as bending) and increased longitudinal twist (also referred to herein as twisting) relative to the native configuration. Such increased curvature results at least in part due to a combination of features including the threaded and tapered head portion 80 of fasteners 70 is tapered, the fastener apertures 630 in the bone plate 610 are undersized relative to the fasteners 70, and the fasteners 70 are made from a material harder than the material in which the apertures 630 are formed so that the installed fasteners 70 volumetrically displace bone plate material defining the apertures 630. As a result, threadably inserting the threaded heads 80 of the fasteners into fastener apertures 630 volumetrically displaces portions of the polymeric material of the bone plate in a manner to induce the bone plate to transition from the native configuration to the multi-curved configuration.

Additional features of bone plate 610 increase the ability of bone plate 610 to experience induced curvatures when the oversized fasteners 70 are inserted into bone plate 610. Bone plate 610 comprises fastener pads 631 in which fastener apertures 630 are formed. Fastener pads 631 are connected by beam sections 632. Additional features of beam sections 632 reduce the amount of beam material, thus making it easier to induces curvature in beam sections 632. Beam sections 632 include dimples 635, which reduce the size of beam sections 632. As depicted, dimples 635 are adjacent to the posterior side of bone plate 610. In other embodiments, dimples 635 are adjacent to the anterior side 660 of bone plate 610. In various embodiments, dimples 635 may appear on one or both sides of beam sections 632.

Installation of fasteners in fastener apertures 630 of bone plate 610 results in a change of configuration of bone plate 610 between the native configuration of FIGS. 8A and 8B to an induced multi-curved configuration. The installation of fasteners results in increased out of plane radius in the negative Z direction, that is, an increased out of plane radius curvature relative to the native configuration. The installation of fasteners also results in increased longitudinal twist of bone plate 610 around the X axis, that is, an increased longitudinal twist relative to the native configuration. It is noted that the induced configuration change results primarily due to the installation of the fasteners into apertures 630 of bone plate 610. This change in configuration happens even if the fasteners 70 are inserted through the bone plate into open air or through bone plate 610 into underlying bone portions (not shown). This shows that a main factor contributing to the change in configuration is due to the manner by which the fasteners engage and exert bending and twisting forces in the bone plate 610.

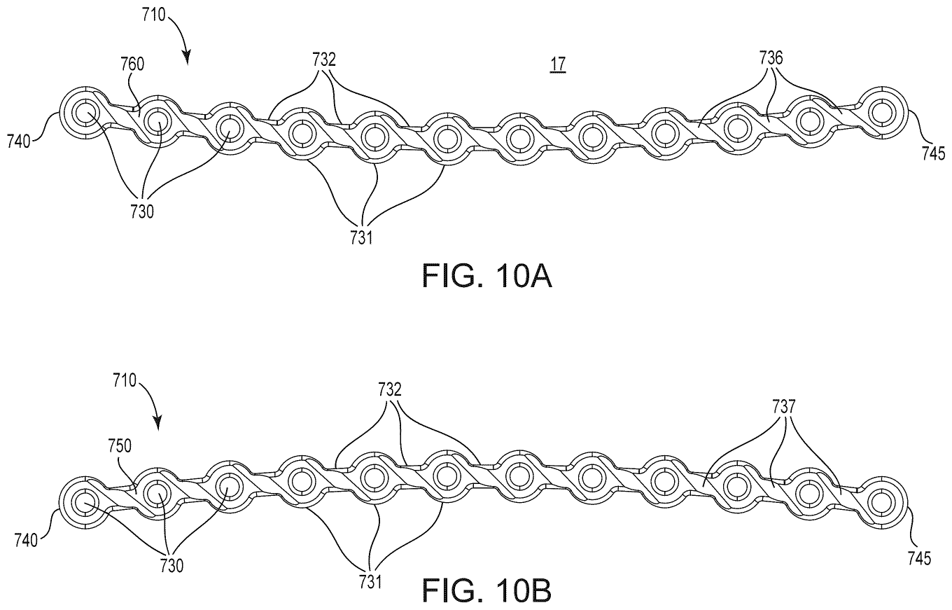

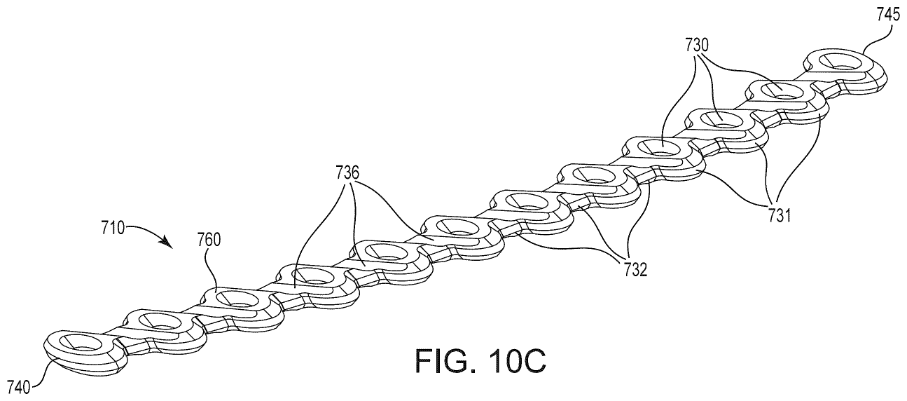

FIGS. 10A, 10B, and 10C show a further embodiment of a surgical bone plate 710 of the present invention useful to help fixate at least first and second underlying bone portions (not shown), such as may occur when a bone (not shown) has been broken or fractured. A corresponding surgical bone plate system includes surgical bone plate 710 and a plurality of fasteners such as fasteners 70 of FIGS. 2A, 2B, 2C, and 5.

The bone plate 710 and fasteners such as fasteners 70 of FIGS. 2A-C, 3, and 5 are configured so that insertion of fasteners 70 into the bone plate 710 causes increased curvature of the bone plate 710 with respect to at least two of bending, twisting, and lateral curvature, preferably including at least increased bending curvature and increased twisting curvature. This increased curvature helps to conform the bone plate to the geometry of the underlying bone tissue. This allows, if desired, bone plate 710 to be in an initial configuration before fasteners 70 are installed that is substantially planar. This allows for easy storage and packaging, as the induced curvature is not yet present until the fasteners are installed. The geometry of the bone plate 710 can them be adapted and customized to the geometry of the underlying bone tissue when the fasteners are installed.

Moreover, the degree of curvature of bone plate 710 can be tuned depending on how much torque is used to install the fasteners. For example, in illustrative embodiments, the degree of bending curvature of bone plate 710 can be adjusted from a range of 0 m⁻¹ to 20 m⁻¹, preferably 2 m⁻¹ to 15 m⁻¹, more preferably from 2 m⁻¹ to 12 m⁻¹ depending on the torque used to install each of the fasteners. Generally, using more torque tends to induce greater curvature, while using less torque tends to induce lesser curvature. The torque can be varied from fastener to fastener so that the degree of curvature can be tuned to greater and lesser degrees along the length of bone plate 710.

FIG. 10A shows a top view of bone plate 710 according to the present invention in its native configuration. The native configuration refers to the configuration that the bone plate 710 naturally assumes when set down onto a flat surface 17 with posterior (bottom) surface 750 facing downward against the supporting surface and the anterior (top) surface 760 facing upward. In surgical use, the posterior surface would be placed facing toward the underlying bone, while the anterior surface 760 would face upward away from the bone. In the native configuration, there are no fasteners installed in the bone plate 710. Other than gravity and the supporting force of the flat surface acting on the bone plate 710, no other forces are acting on the bone plate 710 such as to stretch or compress the bone plate 710 or to cause lesser or greater curvature than the bone plate 710 assumes on its own accord.

Bone plate 710 has at least one longitudinal twist inducing characteristic such that installation of fasteners, such as fasteners 70 of FIGS. 2A-C, and 5, into the bone plate 710 will induce increased twisting curvature as well as increased bending curvature. As can be seen best in FIG. 10A, bone plate 710 is formed with an initial degree of in-plane radius, or lateral, curvature in the native configuration. The presence of this curvature helps to induce increased twisting curvature in bone plate 710 when fasteners are installed in the bone plate 710. Any suitable curvature profile may be employed, for example, circular, elliptical, parabolic, logarithmic, or any other curve. The curvature profile may be symmetric or asymmetric. The bone plate may incorporate multiple curved sections (not shown) that meet at inflection points.

The degree of pre-formed lateral curvature may vary over a wide range. In some embodiments, the degree of pre-formed lateral curvature is in the range from 0.5 m⁻¹ to 12 m⁻¹, preferably 0.5 m⁻¹ to 8 m⁻¹, or even 1 m⁻¹ m to 8 m⁻¹.

Bone plate 710 may be formed from a wide variety of materials. Preferably, bone plate 710 is formed from one or more biocompatible polymers. One or more biocompatible polymers are useful, as using these to form the bone plate 710, particularly in regions including fastener apertures 730, allows the fasteners to volumetrically displace the bone plate material when the screws are inserted into the bone plate 710. In practical effect, the apertures 730 are undersized relative to the fasteners so that the fasteners not only threadably engage the biocompatible polymeric material, but also forcibly push the material aside in order to allow the apertures 730 to increase sufficiently in size to allow the fasteners to be inserted through the apertures 730 and screwed into the underlying bone tissue. These volumetric displacement forces are believed to be a key reason as to why insertion of the fasteners into the bone plate 710 induces increased bending and twisting curvature in the bone plate 710.

Examples of biocompatible polymer materials useful for forming bone plate 710 include one or more of PEEK (Polyether Ether Ketone), PLLA (Poly-L-Lactic Acid), PGA (Polyglycolic Acid), PVC (polyvinyl chloride), PE (polyethelene), PP (polypropylene), PLA (Polylactic Acid), PTFE (Polytetrafluoroethylene), PMMA (Polymethyl Methacrylate), silicone, PTMC (polytrimethylcarbonate), PVDF (Polyvinylidene Fluoride), UHMWPE (Ultra-High-Molecular-Weight Polyethylene) and combinations of these. PEEK is a preferred biocompatible polymer for use in bone plate 710.

Bone plate 710 comprises a plurality of fastener apertures 730. In a preferred mode of practice as shown, the apertures are arranged in a 1×n aperture array extending along a length of the bone plate in a direction from a first bone plate end 740 to a second bone plate end 745, where n is the number of apertures and n is at least 6 and preferably is in the range from 6 to 50. If n is below 6, the array of apertures could be ineffective at helping to induce twisting curvature when fasteners are installed. For many instances, n may be in the range from 6 to 50, preferably 8 to 50, more preferably 8 to 40, or even 8 to 25 or even 9 to 25 fastener apertures. For purposes of illustration, FIGS. 10A-C show an illustrative embodiment of bone plate 710 having twelve fastener apertures 730 spaced between first bone plate end 740 and second bone plate end 745.

The length of bone plate 710 may vary over a wide range, depending on the length of the bone being treated and its injury (ies). Illustrative lengths may be in a range from 50 mm to 1000 mm. Illustrative widths may be in a range from 5 mm to 50 mm, and typical thicknesses between 1 mm to 10 mm.

FIGS. 10A-C depict an embodiment of a bone plate 710 which, in its depicted native configuration, has no out of plane radius curvature and no longitudinal twist curvature, but which has in-plane radius curvature. This embodiment of bone plate 710, thus, is planar. The pre-formed, lateral curvature of bone plate 710 helps to induce increased twisting curvature when fasteners are installed on bone plate 710. The installation of the fasteners also induces increased bending curvature as well. The practical effect of this is that installation of the fasteners in bone plate 710 and the underlying bone helps to induce curvature that, in practical effect, functions as pre-formed lateral and bending curvature in bone plate 710.

The bone plate system according to the present disclosure includes a plurality of fasteners, such as fasteners 70 of FIGS. 2A-C, 3, and 5, configured to be installed in the fastener apertures 730 of the bone plate 710 to couple the bone plate 710 to underlying bone portions (not shown) being fixated. An illustrative configuration of fasteners 70 is shown in FIG. 5 and is discussed above.

The bone plate 710 has a native configuration and an induced multi-curved configuration (not shown). The induced multi-curved configuration is characterized by increased out of plane curvature (also referred to herein as bending) and increased longitudinal twist (also referred to herein as twisting) relative to the native configuration. Such increased curvature results at least in part due to a combination of features including the threaded and tapered head portion 80 of fasteners 70 is tapered, the fastener apertures 730 in the bone plate 710 are undersized relative to the fasteners 70, and the fasteners 70 are made from a material harder than the material in which the apertures 730 are formed so that the installed fasteners 70 volumetrically displace bone plate material defining the apertures 730. As a result, threadably inserting the threaded heads 80 of the fasteners into fastener apertures 730 volumetrically displaces portions of the polymeric material of the bone plate in a manner to induce the bone plate to transition from the native configuration to the multi-curved configuration.

Additional features of bone plate 710 increase the ability of bone plate 710 to experience induced curvatures when the oversized fasteners 70 are inserted into bone plate 710. Bone plate 710 comprises fastener pads 731 in which fastener apertures 730 are formed. Fastener pads 731 are connected by beam sections 732. Additional features of beam sections 732 reduce the amount of force needed to induces curvature in beam sections 732. Beam sections 732 include diagonal anterior scallops 736 appearing on anterior surface 760 and diagonal posterior scallops 737 appearing on posterior surface 750, which reduce the size of beam sections 732. In various embodiments, diagonal anterior scallops 736 and diagonal posterior scallops 737 may appear or be omitted separately on any beam section 732 of bone plate 710.

When fasteners are installed into bone plate 710, a major portion of the volumetric displacement forces act against bone plate regions proximal to the diagonal anterior scallops 736. This interaction helps to promote increased twisting curvature. Lesser volumetric displacement forces act proximal to the diagonal posterior scallops 737. Consequently, this interaction has less impact on helping to induce increased twisting curvature.

Installation of fasteners in fastener apertures 730 of bone plate 710 results in a change of configuration of bone plate 710 between the native configuration of FIGS. 10A-C to an induced multi-curved configuration. The installation of fasteners results in increased out of plane radius in the negative Z direction, that is, an increased out of plane radius curvature relative to the native configuration. The installation of fasteners also results in increased longitudinal twist of bone plate 710 around the X axis, that is, an increased longitudinal twist relative to the native configuration. It is noted that the induced configuration change results primarily due to the installation of the fasteners into apertures 730 of bone plate 710. This change in configuration happens even if the fasteners 70 are inserted through the bone plate into open air or through bone plate 710 into underlying bone portions (not shown). This shows that a main factor contributing to the change in configuration is due to the manner by which the fasteners engage and exert bending and twisting forces in the bone plate 710.

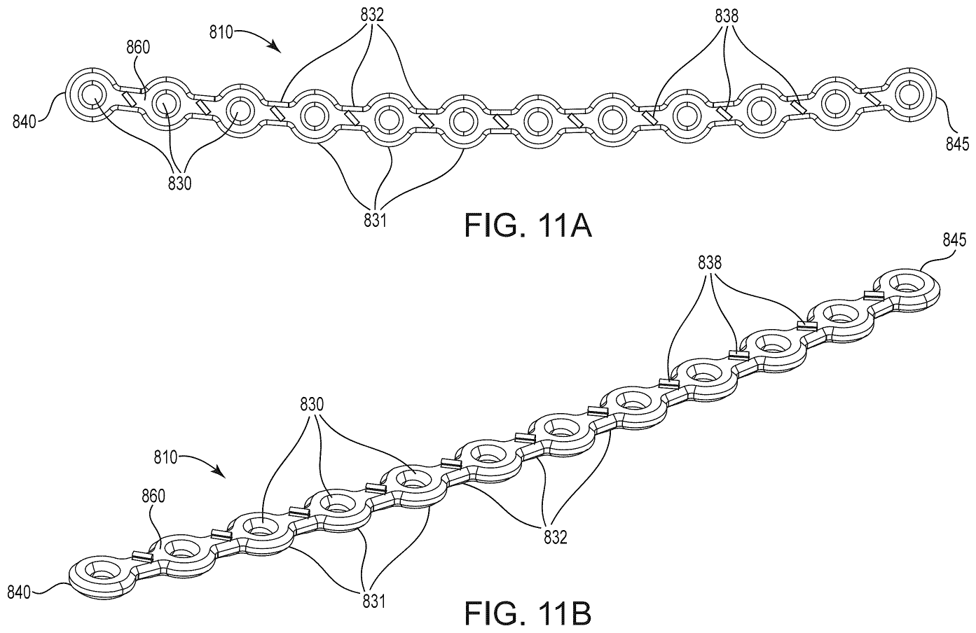

FIGS. 11A & 11B show a further embodiment of a surgical bone plate 810 of the present invention useful to help fixate at least first and second underlying bone portions (not shown), such as may occur when a bone (not shown) has been broken or fractured. A corresponding surgical bone plate system includes surgical bone plate 810 and a plurality of fasteners such as fasteners 70 of FIGS. 2A, 2B, 2C, and 5.

The bone plate 810 and fasteners such as fasteners 70 of FIGS. 2A-C, and 5 are configured so that insertion of fasteners into the bone plate 810 causes increased curvature of the bone plate with respect to at least two of bending, twisting, and lateral curvature, preferably including at least increased bending curvature and increased twisting curvature. This increased curvature helps to conform the bone plate to the geometry of the underlying bone tissue. This allows, if desired, bone plate 810 to be in an initial configuration before fasteners 70 are installed that is substantially planar. This allows for easy storage and packaging, as the induced curvature is not yet present until the fasteners are installed. The geometry of the bone plate can them be adapted and customized to the geometry of the underlying bone tissue when the fasteners are installed.

Moreover, the degree of curvature of bone plate 810 can be tuned depending on how much torque is used to install the fasteners. For example, in illustrative embodiments, the degree of bending curvature of bone plate 810 can be adjusted from a range of 0 m⁻¹ to 20 m⁻¹, preferably 2 m⁻¹ to 15 m⁻¹, more preferably from 2 m⁻¹ to 12 m⁻¹ depending on the torque used to install each of the fasteners. Generally, using more torque tends to induce greater curvature, while using less torque tends to induce lesser curvature. The torque can be varied from fastener to fastener so that the degree of curvature can be tuned to greater and lesser degrees along the length of bone plate 810.

FIG. 11A shows a top view of bone plate 810 according to the present invention in its native configuration. The native configuration refers to the configuration that the bone plate 810 naturally assumes when set down onto a flat surface 17 with posterior (bottom) surface (not visible) facing downward against the supporting surface and the anterior (top) surface 860 facing upward. In surgical use, the posterior surface would be placed facing toward the underlying bone, while the anterior surface 860 would face upward away from the bone. In the native configuration, there are no fasteners installed in the bone plate 810. Other than gravity and the supporting force of the flat surface acting on the bone plate 810, no other forces are acting on the bone plate 810 such as to stretch or compress the bone plate 810 or to cause lesser or greater curvature than the bone plate 810 assumes on its own accord.

Bone plate 810 has at least one longitudinal twist inducing characteristic such that installation of fasteners, such as fasteners 70 of FIGS. 2A-C, and 5, into the bone plate 810 will induce increased twisting curvature. As can be seen best in FIG. 11A, bone plate 810 is formed with an initial degree of in-plane radius, or lateral, curvature in the native configuration. The presence of this curvature helps to induce increased twisting curvature in bone plate 810 when fasteners are installed in the bone plate 810. Any suitable curvature profile may be employed, for example, circular, elliptical, parabolic, logarithmic, or any other curve. The curvature profile may be symmetric or asymmetric. The bone plate may incorporate multiple curved sections (not shown) that meet at inflection points.

The degree of pre-formed lateral curvature may vary over a wide range. In some embodiments, the degree of pre-formed lateral curvature is in the range from 0.5 m⁻¹ to 12 m⁻¹, preferably 0.5 m⁻¹ to 8 m⁻¹, or even 1 m⁻¹ m to 8 m⁻¹.

Bone plate 810 may be formed from a wide variety of materials. Preferably, bone plate 810 is formed from one or more biocompatible polymers. One or more biocompatible polymers are useful, as using these to form the bone plate 810, particularly in regions including fastener apertures 830, allows the fasteners to volumetrically displace the bone plate material when the screws are inserted into the bone plate 810. In practical effect, the apertures 830 are undersized relative to the fasteners so that the fasteners not only threadably engage the biocompatible polymeric material, but also forcibly push the material aside in order to allow the apertures 830 to increase sufficiently in size to allow the fasteners to be inserted through the apertures 830 and screwed into the underlying bone tissue. These volumetric displacement forces are believed to be a key reason as to why insertion of the fasteners into the bone plate 810 induces increased bending and twisting curvature in the bone plate 810.

Examples of biocompatible polymer materials useful for forming bone plate 810 include one or more of PEEK (Polyether Ether Ketone), PLLA (Poly-L-Lactic Acid), PGA (Polyglycolic Acid), PVC (polyvinyl chloride), PE (polyethelene), PP (polypropylene), PLA (Polylactic Acid), PTFE (Polytetrafluoroethylene), PMMA (Polymethyl Methacrylate), silicone, PTMC (polytrimethylcarbonate), PVDF (Polyvinylidene Fluoride), UHMWPE (Ultra-High-Molecular-Weight Polyethylene) and combinations of these. PEEK is a preferred biocompatible polymer for use in bone plate 810.

Bone plate 810 comprises a plurality of fastener apertures 830. In a preferred mode of practice as shown, the apertures are arranged in a 1×n aperture array extending along a length of the bone plate in a direction from a first bone plate end 840 to a second bone plate end 845, where n is the number of apertures and n is at least 6 and preferably is in the range from 6 to 50. If n is below 6, the array of apertures could be ineffective at helping to induce twisting curvature when fasteners are installed. For many instances, n may be in the range from 6 to 50, preferably 8 to 50, more preferably 8 to 40, or even 8 to 25 or even 9 to 25 fastener apertures. For purposes of illustration, FIGS. 11A & 11B show an illustrative embodiment of bone plate 810 having thirteen fastener apertures 830 spaced between first bone plate end 840 and second bone plate end 845.

The length of bone plate 810 may vary over a wide range, depending on the length of the bone being treated and its injury (ies). Illustrative lengths may be in a range from 50 mm to 1000 mm. Illustrative widths may be in a range from 5 mm to 50 mm, and typical thicknesses between 1 mm to 10 mm.

FIGS. 11A and 11B depict an embodiment of a bone plate 810 which, in its native configuration, has no out of plane radius curvature and no longitudinal twist curvature, but which has in-plane radius curvature. This embodiment of bone plate 810 in the native configuration, thus, is planar. The pre-formed, lateral curvature of bone plate 810 helps to induce increased twisting curvature when fasteners are installed on bone plate 810. The installation of the fasteners also induces increased bending curvature as well. The practical effect of this is that installation of the fasteners in bone plate 810 and the underlying bone helps to induce curvature that, in practical effect, functions as pre-formed lateral and bending curvature in bone plate 810.

The bone plate system according to the present disclosure includes a plurality of fasteners, such as fasteners 70 of FIGS. 2A-C, 3, and 5, configured to be installed in the fastener apertures 830 of the bone plate 810 to couple the bone plate 810 to underlying bone portions (not shown) being fixated. An illustrative configuration of fasteners 70 is shown in FIG. 5 and discussed above.

The bone plate 810 has a native configuration and an induced multi-curved configuration (not shown). The induced multi-curved configuration is characterized by increased out of plane curvature (also referred to herein as bending) and increased longitudinal twist (also referred to herein as twisting) relative to the native configuration. Such increased curvature results at least in part due to a combination of features including the threaded and tapered head portion 80 of fasteners 70 is tapered, the fastener apertures 830 in the bone plate 810 are undersized relative to the fasteners 70, and the fasteners 70 are made from a material harder than the material in which the apertures 830 are formed so that the installed fasteners 70 volumetrically displace bone plate material defining the apertures 830. As a result, threadably inserting the threaded heads 80 of the fasteners into fastener apertures 830 volumetrically displaces portions of the polymeric material of the bone plate in a manner to induce the bone plate to transition from the native configuration to the multi-curved configuration.

Additional features of bone plate 810 increase the ability to control induced curvature in the bone plate 810 when the oversized fasteners 70 are inserted into bone plate 810. Bone plate 810 comprises fastener pads 831 in which fastener apertures 830 are formed. Fastener pads 831 are connected by beam sections 832. Stiffening features 838 are borne diagonally on the anterior surface 860 of beam sections 832. Stiffening features 838 tend to resist induced curvature in bone plate 810. However, stiffening features 838 are removable, for example by the surgeon, during installation of bone plate 810. This allows the fit of bone plate to be more easily customized. If greater amounts of induced curvature are desired in any one or more regions of bone plate 810, the stiffening ribs 838 can be removed so that the bone plate 810 will more easily be curved in those regions. Advantageously, therefore, by allowing any given stiffening feature 838 to remain, or by partially or wholly removing any given stiffening feature 838, the surgeon may adjust the curvature of bone plate 810 in a given location along bone plate 810

Installation of fasteners in fastener apertures 830 of bone plate 810 results in a change of configuration of bone plate 810 between the native configuration of FIGS. 8A and 8B to an induced multi-curved configuration. The installation of fasteners results in increased out of plane radius in the negative Z direction, that is, an increased out of plane radius curvature relative to the native configuration. The installation of fasteners also results in increased longitudinal twist of bone plate 810 around the X axis, that is, an increased longitudinal twist relative to the native configuration. It is noted that the induced configuration change results primarily due to the installation of the fasteners into apertures 830 of bone plate 810. This change in configuration happens even if the fasteners 70 are inserted through the bone plate into open air or through bone plate 810 into underlying bone portions (not shown). This shows that a main factor contributing to the change in configuration is due to the manner by which the fasteners engage and exert bending and twisting forces in the bone plate 810.

All patents, patent applications, and publications cited herein are incorporated herein by reference in their respective entities for all purposes. The foregoing detailed description has been given for clarity of understanding only. No unnecessary limitations are to be understood therefrom. Various modifications and alterations of this disclosure will become apparent to those skilled in the art without departing from the scope and principles of this disclosure, and it should be understood that this disclosure is not to be unduly limited to the illustrative embodiments set forth hereinabove.

We claim:

1. A surgical bone plate system useful to help fixate at least first and second underlying bone portions, comprising:
a) a polymeric bone plate comprising at least one biocompatible polymer material, wherein the polymeric bone plate comprises a native configuration and an induced multi-curved configuration, and wherein the induced multi-curved configuration includes increased curvature comprising at least two of increased bending curvature, increased twisting curvature, and/or increased lateral curvature relative to the native configuration;
b) a 1×n aperture array of fastener apertures formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is 6 or more; and
c) a plurality of fasteners that are configured to be installed in the fastener apertures and into the at least first and second underlying bone portions in a manner effective to couple the polymeric bone plate to the at least first and second underlying bone portions; and
wherein the fastener apertures are undersized relative to the fasteners and wherein the fasteners comprises a fastener material that is harder than the at least one biocompatible polymer material in which the fastener apertures are formed such that inserting the fasteners into the fastener apertures of the plurality of fastener apertures volumetrically displaces the at least one biocompatible polymer in a manner to induce the polymeric bone plate to transition from the native configuration to the multi-curved configuration.

2. The bone plate system according to claim 1, wherein each fastener aperture of the plurality of fastener apertures is provided at a region of the polymeric bone plate having an associated plate thickness, wherein each fastener aperture of the plurality of fastener apertures has a plate aperture diameter, wherein each fastener of said plurality of fasteners has a fastener head diameter, wherein each fastener of the plurality of fasteners and the corresponding aperture have a volumetric displacement, and wherein at least one fastener and a corresponding fastener aperture of the plurality of fastener apertures has at least one volume displacement characteristic in a manner such that threadably inserting the threaded and tapered head of the at least one fastener into the corresponding fastener aperture volumetrically displaces a plurality of portions of the at least one biocompatible polymer in a manner to induce the polymeric bone plate to transition from the native configuration to the multi-curved configuration, wherein the volumetric displacement characteristic is selected from one or more ratios of the group consisting of:
i) a ratio of volumetric displacement to plate thickness in the range from 0.55 mm² to 1.2 mm²;
ii) a ratio of volumetric displacement to plate aperture diameter in the range from 0.45 mm² to 1.2 mm²; and
iii) a ratio of volumetric displacement to screw head diameter in the range from 0.30 mm² to 0.60 mm².

3. The bone plate system according to claim 2 wherein the volumetric displacement relationship includes i).

4. The bone plate system according to claim 2 wherein the volumetric displacement relationship includes ii).

5. The bone plate system according to claim 2 wherein the volumetric displacement relationship includes iii).

6. The bone plate system according to claim 2, wherein the volumetric displacement relationship includes at least two of i), ii), and/or iii).

7. The bone plate system according to claim 2, wherein the volumetric displacement relationship includes i), ii), and iii).

8. The bone plate system according to claim 1, wherein the at least first and second underlying bone portions comprise first and second rib bone portions.

9. The bone plate system according to claim 1, wherein the increased curvature comprises increased bending curvature and increased twisting curvature.

10. The bone plate system according to claim 1, wherein the polymeric bone plate comprises lateral curvature in the native configuration.

11. The bone plate system according to claim 1 wherein the bone plate has a posterior surface and an anterior surface and wherein volumetric displacement increases monotonically from the posterior surface to the anterior surface.

12. The bone plate system according to claim 1 wherein the bone plate has an in-plane radius curvature in a range from 2 m⁻¹ to 20 m⁻¹ in its native configuration.

13. The bone plate system according to claim 1 wherein n is in the range from 8 to 25.

14. The bone plate system according to claim 1, wherein the bone plate comprises PEEK and at least one of the plurality of fasteners comprises titanium.

15. The bone plate system according to claim 1, wherein the bone plate comprises a plurality of pads and a plurality of beam sections that diagonally interconnect the pads, and wherein at least a portion of the plurality of fastener apertures are formed in the pads.

16. The bone plate system of claim 15, wherein at least one beam section includes an elongate window.

17. The bone plate system according to claim 1, wherein the bone plate comprises a plurality of pads and a plurality of beam sections that interconnect the pads, wherein at least a portion of the plurality of fastener apertures are formed in the pads, and wherein at least one beam section includes dimples formed on a posterior side of the beam section.

18. The bone plate system according to claim 1, wherein the bone plate comprises a plurality of pads and a plurality of beam sections that interconnect the pads, wherein at least a portion of the plurality of fastener apertures are formed in the pads, and wherein at least one beam section includes a diagonal scallop.

19. The bone plate system according to claim 18, wherein the at least one beam section including the diagonal scallop has an anterior side and a posterior side, and wherein the scallop is formed on the anterior side.

20. The bone plate system according to claim 18, wherein the at least one beam section including the diagonal scallop has an anterior side and a posterior side, and wherein the scallop is formed on the posterior side.

21. The bone plate system according to claim 18, wherein the at least one beam section including the diagonal scallop has an anterior side and a posterior side, wherein the diagonal scallop is formed on the posterior side, and wherein the at least one beam section including the diagonal scallop further comprises an additional scallop on the anterior side.

22. The bone plate system according to claim 1, wherein the bone plate comprises a plurality of pads and a plurality of beam sections that interconnect the pads, wherein at least a portion of the plurality of fastener apertures are formed in the pads, and wherein at least one beam section includes a diagonal rib projecting from an anterior surface of the at least one beam section.

23. A surgical bone plate system useful to help fixate at least first and second underlying bone portions, comprising:
d) a polymeric bone plate comprising at least one biocompatible polymer material, wherein the polymeric bone plate comprises a native configuration and an induced multi-curved configuration, and wherein the induced multi-curved configuration includes increased curvatures comprising at least two of increased bending curvature, increased twisting curvature, and/or increased lateral curvature relative to the native configuration;
e) a 1×n aperture array formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is in the range from 6 to 50, wherein each fastener aperture of the plurality of fastener apertures is provided at a region of the polymeric bone plate having an associated plate thickness, and wherein each fastener aperture of the plurality of fastener apertures has a plate aperture diameter; and
f) a plurality of fasteners that are configured to be installed in the fastener apertures and in the at least first and second underlying bone portions in a manner effective to couple the polymeric bone plate to the at least first and second underlying bone portions, wherein each fastener of the plurality of fasteners has a head diameter, wherein each fastener of the plurality of fasteners is oversized relative to a corresponding aperture of the plurality of fastener apertures, and wherein each fastener of the plurality of fasteners and a corresponding aperture of the plurality of fastener apertures have a volumetric displacement relationship that is selected from one or more ratios in the group consisting of:
4) a ratio of volumetric displacement to plate thickness in the range from 0.55 to 1.2;
5) a ratio of volumetric displacement to plate aperture diameter in the range from 0.45 to 1.2; and/or
6) a ratio of volumetric displacement to fastener head diameter in the range from 0.30 to 1.00.

24. A bone plate system useful to help fixate at least first and second underlying bone portions, comprising:
a) a polymeric bone plate comprising at least one biocompatible polymer material, wherein the polymeric bone plate comprises lateral curvature in a native configuration;
b) a 1×n aperture array of fastener apertures formed in the at least one biocompatible polymer material of the polymeric bone plate and extending along a length of the polymeric bone plate in a direction from a first bone plate end to a second bone plate end, wherein the 1×n aperture array comprises n fastener apertures formed in the at least one biocompatible polymer material, and wherein n is 6 or more; and
c) a plurality of metal fasteners that are configured to be installed in the fastener apertures and into the at least first and second underlying bone portions in a manner effective to couple the polymeric bone plate to the at least first and second underlying bone portions, wherein each metal fastener of the plurality of metal fasteners is oversize relative to a corresponding fastener aperture of the plurality of fastener apertures.
